(19) United States
(12) Patent Application Publication
(10) Pub. No.: US 2026/0115359 A1
(43) Pub. Date: Apr. 30, 2026

(54) TISSUE-DERIVED IMPLANT USEFUL FOR CARTILAGE REGENERATION

(71) Applicants: City University of Hong Kong, Hong Kong (CN); Peking University Third Hospital, Beijing (CN)

(72) Inventors: Dong SUN, Hong Kong (CN); Hanjin HUANG, Hong Kong (CN); Junyang LI, Hong Kong (CN); Cheng WANG, Beijing (CN)

(21) Appl. No.: 18/932,725

(22) Filed: Oct. 31, 2024

(51) Int. Cl. A61L 27/36; A61L 27/04; A61L 27/38; A61N 2/00

(52) U.S. Cl. CPC A61L 27/3612; A61L 27/04; A61L 27/3683; A61L 27/3834; A61N 2/004; A61L 2430/06

(57) ABSTRACT

A tissue-derived implant including: a plurality of decellularized extracellular cartilage particles, wherein each of the plurality of cartilage extracellular particles comprise one or more magnetic particles and mesenchymal stem cells and method of preparation and method use thereof. The tissue-derived implant can be guided to the site of a cartilage defect after administration by use of a magnetic field.

### STATEMENT REGARDING PRIOR DISCLOSURES BY THE INVENTORS OR JOINT INVENTORS UNDER 37 CFR 1.77(b)(6)

Part of the present invention was disclosed in a paper published in Hanjin Huang, et al., Using Decellularized Magnetic Microrobots to Deliver Functional Cells for Cartilage Regeneration, Nano-Micro Small, Volume 20, Issue 11, 2304088, 2024. This paper is a grace period inventor-originated disclosure disclosed within one year before the filing date of this application and falls within the exceptions defined under 35 USC § 102(b)(1). This paper is hereby incorporated by reference in its entirety.

### TECHNICAL FIELD

The present disclosure relates to a magnetic tissue-derived implant useful for treating cartilage defects and methods of use thereof.

### BACKGROUND

Cartilage serves as the central protective cushion between bones, acting as a shock absorber and stabilizer that allows free motion. However, factors, such as aging, being overweight, accidents, and chronic or acute diseases, promote the development of defects in the superficial articular cartilage, leading to progressive deterioration and eventual onset of osteoarthritis (OA). This debilitating condition currently affects 7% of the global population, and this number is predicted to increase over the next 30 years ^{[1]}. Despite its critical function, articular cartilage has limited intrinsic healing capacity because of its low vascularity; therefore, its recovery largely depends on therapeutic interventions ^{[2-4]}. The low cellularity and limited regenerative capacity of articular cartilage have attracted the attention of researchers worldwide, but current clinical treatments for OA primarily focus on managing symptom, reducing pain, and delaying the need for surgical joint replacement and do not address cartilage defects. To date, OA has no cure ^{[5]}.

Adult mesenchymal stem cells (MSCs) have been studied for regenerative applications in OA treatment ^{[6, 7]}. Their ability to differentiate into cells of the chondrogenic lineage hints at their potential as materials that facilitate cartilage regeneration. MSCs can be easily obtained from various adult tissues and expanded without the loss of multipotency ^{[8]}. The in vivo therapeutical effects of OA treatments have been reported ^{[9-11]}. However, direct MSCs injection poses challenges, including phenotype loss and undesired differentiation direction due to inappropriate biophysical cues like in an autologous chondrocyte transplantation ^{[12]}. Therefore, embedding MSCs into scaffolds during OA treatment is a necessary process to overcome these problems.

Tissue engineering offers promising prospects for the treatment of OA ^{[13]}. An eligible scaffold should possess a porous structure, biodegradability, nutrient permeability, limited immune rejection, and ability to promote cell migration, proliferation, and differentiation. Freed^{[14]} firstly introduced microcarrier culture technology for chondrocytes. MSCs proliferate and differentiate into chondrocytes in microcarrier culture ^{[15, 16]} and numerous synthetic and natural materials have been considered for use as scaffolds, including poly(lactic-co-glycolic acid) ^{[17]}, fibrin ^{[18]}, collagen ^{[19]}, hyaluronan ^{[20]}, and plasma ^{[21]}. Apart from synthetic scaffolds, natural scaffolds have been discussed because of their inherent complex structure, favorable biochemical composition, high biocompatibility, and biodegradability ^{[9, 22-24]}. Cartilage ECM has attracted interest because it promotes MSC chondrogenic differentiation ^{[9, 25, 26]} and cartilage ECM fragments can accelerate cartilage repair even without cells ^{[27, 28]}.

Magnetic-driven drug delivery has been widely applied for diagnostic and therapeutic purposes, because it enables drugs to penetrate deep tissues and is clinically safe ^{[29-33]}. Coating and actuating strategies are critical factors in designing reliable magnetic-driven drug delivery systems. Magnetic nanoparticles can exhibit biocompatibility, biodegradability, and excellent magnetic properties ^{[34, 15]}. They have been applied to rare cell isolation ^{[36]}, cancer cell-targeted drug delivery ^{[37]}, and peripheral nerve regeneration ^{[38]}. For actuating systems, rotating magnetic field (RMF) is a merging technique independent of visual feedback and has inherent merit in microrobot aggregation ^{[29, 39, 40]}; therefore, microrobots actuated by RMF potentially benefits cell delivery in vivo.

There is thus a need for improved implants useful for treating cartilage defects that address or overcome at least some of the disadvantages in the art.

### SUMMARY

For the first time, we bridged the gap between decellularized scaffolds and magnetic-driven cell delivery. An overview of the process for preparing the tissue-derived implants described herein is shown in FIG. 1. Described herein is the design of decellularized microrobots as products of decellularization and magnetization to achieve in vitro transport and deliver targeted cells for cartilage regeneration. Fresh porcine articular cartilage fragments were pulverized into particles, and particles with a median diameter of 238±62 μm were obtained through sieving. Natural cartilage ECM was decellularized through a series of chemical treatments to maintain sufficient structural and biochemical components and ultrastructure disruption to the native microenvironment was minimized. The scaffolds were magnetized through dip coating in 200 nm magnetite nanoparticles (Fe₃O₄ NPs). To achieve targeted delivery, RMFs were applied as magnetic actuation. All the experiments showed that compared with most traditional passive drug-delivery microcarriers, the design enhanced the cell-carrying capacity and provided robust mobility for the microcarriers. To verify successful cell removal, we carried out DNA quantification. With the use of scanning electron microscopy (SEM), surface and pore architecture were tested, demonstrating that microcarriers maintain the naturally porous structure and the components of cartilage. Bone marrow MSCs were selected as cellular drugs to treat OA because of their various differentiation abilities, including chondrocytes. Fluorescence imaging and statistical analysis performed on MSCs showed that microrobots exhibited high cell loading ability. Cytotoxicity and biodegradability tests indicated they had low cytotoxicity and can degrade rapidly. Experiments on 2D substrates and 3D real-cartilage surfaces also demonstrated that our microrobots loaded with cells could be actuated, aggregated, and precisely transported to a desired position through a self-constructed electromagnetic coil setup. Furthermore, cells loaded in the microrobots can be automatically released by conducting in vitro experiments. All the results showed that this cell carrier design can successfully carry and deliver targeted cells to desired locations. It offers promising prospects as a tool for minimally invasive articular cartilage regeneration.

In a first aspect, provided herein is a tissue-derived implant comprising: a plurality of decellularized extracellular cartilage particles, wherein each of the plurality of cartilage extracellular particles comprise one or more magnetic particles and mesenchymal stem cells (MSCs).

In certain embodiments, the plurality of decellularized extracellular cartilage particles have an average diameter of 100-400 μm.

In certain embodiments, the plurality of decellularized extracellular cartilage particles have an average diameter about 238 μm.

In certain embodiments, the plurality of decellularized extracellular cartilage particles are derived from human, mouse, rat, dog, cat, rabbit, horse, pig, or nonhuman primate cartilage.

In certain embodiments, the plurality of decellularized extracellular cartilage particles are prepared by contacting a plurality of extracellular cartilage particles with a solution comprising sodium dodecyl sulfate thereby forming the plurality of decellularized extracellular cartilage particles.

In certain embodiments, the one or more magnetic particles have an average size of 100-300 nm.

In certain embodiments, the one or more magnetic particles have an average size about 200 nm.

In certain embodiments, the one or more magnetic particles comprise Fe₂O₃, Fe₃O₄, FeNi, FePt, Fe, or CoNi alloy.

In certain embodiments, the one or more magnetic particles have an average coating concentration on the plurality of decellularized extracellular cartilage particles of 2-20% wt/wt based on the weight of the one or more magnetic particles and a wet weight of the decellularized extracellular cartilage particles.

In certain embodiments, the one or more magnetic particles have an average coating concentration on the plurality of decellularized extracellular cartilage particles of about 10‰ wt/wt based on the weight of the one or more magnetic particles and a wet weight of the decellularized extracellular cartilage particles.

In certain embodiments, the MSCs comprise bone marrow mesenchymal stem cells.

In certain embodiments, the MSCs are present on a surface of each of the plurality of decellularized extracellular cartilage particles at seeding density of 300-3,000 MSCs per mm² of the plurality of decellularized extracellular cartilage particles.

In certain embodiments, the plurality of decellularized extracellular cartilage particles have an average diameter of 150-350 μm; the one or more magnetic particles comprise Fe₃O₄ and have average diameter of 100-300 nm; and the MSCs comprise bone marrow mesenchymal stem cells.

In certain embodiments, the plurality of decellularized extracellular cartilage particles have an average diameter about 238 μm; the one or more magnetic particles comprise Fe₃O₄ and have average diameter of about 200 nm; and the MSCs comprise bone marrow mesenchymal stem cells.

In certain embodiments, the MSCs are present on a surface of each the plurality of decellularized extracellular cartilage particles at seeding density of 300-3,000 MSCs per mm² of the plurality of decellularized extracellular cartilage particles.

In a second aspect provided herein is a method of treating a cartilage defect in a subject in need thereof, the method comprising: administering a therapeutically effective amount of the tissue-derived implant described herein to the subject.

In certain embodiments, the method further comprises exposing the tissue-derived implant to a magnetic field thereby directing the tissue derived implant to the cartilage defect after administering the tissue-derived implant to the subject.

In certain embodiments, the magnetic field is a rotating magnetic field.

In certain embodiments, method further comprises imaging the tissue-derived implant using ultrasound-guided photoacoustic imagining.

### BRIEF DESCRIPTION OF THE DRAWINGS

The above and other objects and features of the present disclosure will become apparent from the following description of the disclosure, when taken in conjunction with the accompanying drawings.

FIG. 1. Overview of exemplary method for preparing tissue-derived implants described herein. Fresh porcine articular hyaline cartilages were pulverized, sieved, decellularized, and magnetized to fabricate M-CEDSs. They carried functional cells, like MSCs, to the target places for articular cartilage regeneration.

FIG. 2. Microrobots fabrication. (A) The fresh porcine articular hyaline cartilage was cut and cleaned. (B) After grinding and sieving, the granular natural scaffolds were suspended in PBS for further processing. (C) Raw scaffolds and M-CEDS were compared under an inverted optical microscope (D) Size distribution showed that the scaffold had a medium diameter of 238±62 μm. (E) Shape distribution of CEDSs. Error bars indicate SD. (F) Pore size distribution for scaffolds in three groups, namely, raw scaffolds before decellularization, freshly decellularized scaffolds (Day 0), and decellularized scaffolds (Day 10), was investigated. (G) DNA content was measured with a spectrophotometer, showing 98.36% DNA removal. Error bars indicate SD. *p<0.05, **p<0.01, ***p<0.001, n.s. indicates no significant difference. (H) CEDSs were magnetized, and a permanent magnet demonstrated their magnetized characteristic. (I) Energy dispersive X-ray analysis revealed the carbon, iron element and sodium distribution in the M-CEDSs. Pixels marked in light color are carbon elements, whereas those marked in darker color are iron elements. The rectangle indicates the area shown on the images below at high magnification. Scale bars: 100 μm in (i) and 20 μm in (ii).

FIG. 3. Simulation results of mechanical properties, velocity fields, fluid friction, and mobility. (A) A human sphere, an M-CEDS sphere, a solid sphere, an M-CEDS cube, and a solid cube were modelled for human cartilages, porcine-based M-CEDSs, and commercial microcarriers. (B) Stress distribution during compression processes for mechanical properties. (C) Velocity fields of all structures. (D) Quantitative results for the mechanical property tests. The symbol k means the slope. (E) The resistance forces changed with respect to time for structures. The solid line indicates the magnitude of the magnetic driving force. (F) Mobility tests.

FIG. 4. Cytotoxicity test and degradability test. (A) SEM images of MSCs cultured with magnetite nanoparticles on days 1, 3, and 5. Scale bars: 500 μm. (B) The survival rates of MSCs in 1, 3, and 5 days cultured with CEDSs and M-CEDSs after PI staining. NC: negative control; PC: positive control. (C) The statistical analysis of MSCs cultured for 1, 3, and 5 days (n=3). Cells cultured without any additional material were control groups, whereas those cultured with CEDSs and M-CEDSs were investigated as experimental groups. Error bars indicate SD. (D) The degradation percentages were defined as area ratios between degraded scaffolds and their initial areas, which were calculated on days 3, 10, and 18. *p≤0.05, **p≤0.01, ***p≤0.001, n.s. indicates no significant difference. (E) M-CEDSs degraded gradually in the PBS and 10% NaOH environments. Scale bar: 100 μm.

FIG. 5. Cell seeding on microrobots. (A, B) SEM images of cell-loaded M-CEDSs with a coating concentration of 10‰ on day 3. (A) MSCs and (B) MC3T3-E1 fibroblasts. The rectangle indicates the area shown on the right images at a high magnification. Scale bars: 200 μm in (A1, B1) and 50 μm in (A2, B2). (C) Images of MSCs seeded on microrobots coated with 10‰ magnetite on days 1, 2, and 3. Fluorescence images of cell-loaded microrobots with cell nuclei stained with Hoechst 33342 (left), fluorescence images of microrobots cultured with PI-stained MSCs (middle-left), and phase-contrast bright field of cell-seeded microrobots (middle-right), and merge (right). Scale bars: 100 μm (D) Statical result of MSCs loading density with microrobots coated with 0‰, 2‰, 4‰, 6‰, 8‰, and 10‰ magnetite on days 1, 2, and 3. Error bars indicate SD. *p≤0.05. n.s. indicates no significant difference.

FIG. 6. Mobility test was carried out under a rotating magnetic field. (A) A self-constructed electromagnetic coil carried out all driving tests through rotating magnetic fields. The images showed an aggregational process during the mobility test. M-CEDSs loading MSCs with coating concentrations of 2‰, 4‰, 6‰, 8‰, and 10‰ in magnetic fields with increased strength (23.57, 27.76, and 32.74 mT; input currents: 2.5, 3.0, and 3.5 A) in different fluid environments: PBS, BSA, and HA were measured. The aggregation center was highlighted. (B) Translational speed. (C) Rotational speed. (D) Response rate. Error bars indicate SD. (E) (i-iv) Photographs of translocation. All figures were retrieved from recorded videos: of the aggregation of the M-CEDSs and translocation of the M-CEDSs.

FIG. 7. In vitro M-CEDS MSC delivery and release. (A) The in vitro delivery experiment set. The driving and delivery ability of M-CEDSs loaded with MSCs with a coating concentration of 10‰ in a fixed fresh hyaline cartilage fragment was tested. (B) The covering process of M-CEDSs for defects. (C) The results of coverage with respect to M-CEDS input volume for cartilage defects with different diameters: 1.5, 2.0, and 3.0 mm. All defects had the same depth of 1.5 mm. Error bars indicate SD. (D) The curve fitting of necessary volume for chondral defects. (E) The magnetic driving process of M-CEDSs loaded with MSCs. Position: initial location; Positions b and c: attraction centers formed by magnetic fields.

FIG. 8. In vitro cell-release experiments. (A) MSCs were released from the M-CEDSs onto a pure plastic substrate and proliferated at 3 days of cultivation. (B) A relatively long-time cultivation (20 days) was observed, showing a gradual release as the microrobot was degraded.

FIG. 9. PA imaging of the microrobots. (A) A sketch of the experimental setup. The inferior vena cava (IVC) was obtained by dissection from a rat. The extracted IVC was buried in a chicken breast (depth: 20 mm) as a phantom for US/PA imaging of M-CEDSs in a static fluidic environment. (B) The aggregation process was captured. The rat vessel walls were presented in gray color, whereas the M-CEDSs were presented in darkish color. Owing to external magnetic manipulation, the microrobots aggregated at the aggregation center, showing the ability to aggregate inside animal bodies. (C) The aggregation degree was measured by spread length and intensity of region of interest. Error bars indicate SD.

FIG. 10. In vivo therapeutical effects on cartilage defect rat models. (A) (Left) GFP-BMSCs Visualization through Kodak In Vivo Imaging Systems FX at 3 weeks and (Right) Catwalk for gait analysis at 3 weeks. (i) Blank Control, (ii) Negative Control, (iii) CEDSs, (iv) M-CEDSs, (v) MSC, (vi) CEDSs+MSC, and (vii) M-CEDSs+MSC. Unit for fluorescent images: p·sec⁻¹ cm⁻²sr⁻¹. (B) GFP-positive cell tracking in (i-vii) (Left) Phase-contrast light micrograph and in (Right) fluorescence micrograph (C) paw mean intensity, the 100% value is 157.029 a.u. (D) paw area, the 100% value is 1.743 cm². The results are the means±SD (n>3).

FIG. 11. Processing from a fresh knee joint to granular natural scaffolds. (A-C) Initially a pig fresh knee joint was cleaned and cut with scalpels for further treatment. The fragmentized cartilages were cleaned again and put into an electrical grinder. (D) After grinding, the suspension contained natural scaffolds with different sizes, which were selected using a series of nylon microporous membrane with 300-μm and 100-μm pores, sequentially.

FIG. 12. Appearance of M-CEDSs with different coating concentration. The coating concentrations are 2‰, 4‰, 6‰, 8‰, and 10‰(wet weight ratio).

FIG. 13. Shape distribution of CEDSs and M-CEDSs. According to statical analysis, spherical and cubic shapes dominated the shapes population. (A) Dry CEDSs (B) Wet CEDSs (C) Dry M-CEDSs (D) Wet M-CEDSs

FIG. 14. Magnetite loading amount changes in both static and mobile situations. The M-CEDSs were put into both a static fluidic environment and a mobile environment driven by external magnetic fields continuously. Weight percentage indicates the amount of magnetite nanoparticles falling from the scaffold, which was calculated as the weight ratio between actual and theoretical coated magnetite nanoparticles. The theoretical weight is calculated from 10‰ coating concentration. The static group was tested for 21 days while the mobile group was measured for 0, 25, 50, and 75 minutes.

FIG. 15. Self-constructed electromagnetic coil system. Exemplary electromagnetic coil system used in conjunction with certain embodiments of the method described herein.

FIG. 16. Electromagnetic properties of self-constructed electromagnetic coils. The maximum magnetic field strength and gradient at the center of working area under different input currents were measured. When measuring, all four coils are turned on, forming a rotating electromagnetic field for M-CEDSs delivery.

FIG. 17. Volume curve fitting result. To establish the defect-filling model, M-CEDSs volume necessary for a specific defect diameter was fitted. The high coefficient of determination meant that a significant amount of variance was explained.

FIG. 18. Importance of coating concentration to mobility and loading ability. To compare the significance of coating concentration to mobility and loading ability, the change percentage was calculated. The darker symbols are the mobility test while the lighter symbols are loading ability.

FIG. 19. Parameters for simulation studies. Sizes, densities, pore size distribution, Young's modulus, and Poisson's ratio of four simulators are listed.

FIG. 20. Fluidic environments for mobility test. In mobility tests, M-CEDSs were put inside various fluid environments, including two artificial synovial fluids to simulate the physical properties inside the real articular cavity.

FIG. 21. Group formulation of in vivo experiments. In vivo experiments consist of seven groups: Blank Control, Negative Control, CEDSs, M-CEDSs, MSC, CEDSs+MSC, and M-CEDSs+MSC. A tick means positive whereas a cross is negative for every operation.

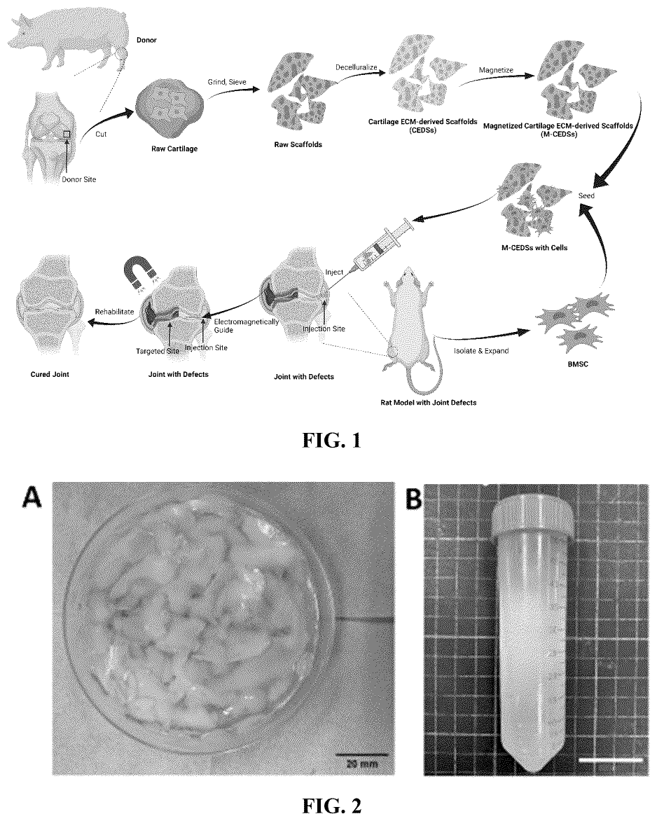

### DETAILED DESCRIPTION

### Definitions

The following terms shall be used to describe the present invention. In the absence of a specific definition set forth herein, the terms used to describe the present invention shall be given their common meaning as understood by those of ordinary skill in the art.

Throughout the present disclosure, unless the context requires otherwise, the word “comprise” or variations such as “comprises” or “comprising”, will be understood to imply the inclusion of a stated integer or group of integers but not the exclusion of any other integer or group of integers. It is also noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the present invention.

Furthermore, throughout the present disclosure and claims, unless the context requires otherwise, the word “include” or variations such as “includes” or “including”, will be understood to imply the inclusion of a stated integer or group of integers, but not the exclusion of any other integer or group of integers.

The use of the singular herein includes the plural (and vice versa) unless specifically stated otherwise. In addition, where the use of the term “about” is before a quantitative value, the present teachings also include the specific quantitative value itself, unless specifically stated otherwise. As used herein, the term “about” refers to a ±10%, ±7%, ±5%, ±3%, ±1%, or ±0% variation from the nominal value unless otherwise indicated or inferred.

As used herein, the term “stem cell” refers to undifferentiated cells having high proliferative potential with the ability to self-renew that can migrate to areas of injury and can generate daughter cells that can undergo terminal differentiation into more than one distinct cell phenotype. These cells have the ability to differentiate into various cells types and thus promote the regeneration or repair of a diseased or damaged tissue of interest.

As used herein, the terms “treat”, “treating”, “treatment”, and the like refer to reducing or ameliorating a disorder/disease and/or symptoms associated therewith. It will be appreciated, although not precluded, treating a disorder or condition does not require that the disorder, condition, or symptoms associated therewith be completely eliminated. In certain embodiments, treatment includes prevention of a disorder or condition, and/or symptoms associated therewith. The term “prevention” or “prevent” as used herein refers to any action that inhibits or at least delays the development of a disorder, condition, or symptoms associated therewith. Prevention can include primary, secondary and tertiary prevention levels, wherein: a) primary prevention avoids the development of a disease; b) secondary prevention activities are aimed at early disease treatment, thereby increasing opportunities for interventions to prevent progression of the disease and emergence of symptoms; and c) tertiary prevention reduces the negative impact of an already established disease by restoring function and reducing disease-related complications.

The term “subject” as used herein, refers to an animal, typically a mammal or a human, that will be or has been the object of treatment, observation, and/or experiment. When the term is used in conjunction with administration of a compound described herein, then the subject has been the object of treatment, observation, and/or administration of the compound described herein. The subject can refer to any animal (e.g., a mammal), including, but not limited to, humans, non-human primates, canines, felines, and rodents.

The term “therapeutically effective amount” as used herein, means that amount of a composition (e.g., the tissue-derived implant) that elicits a biological and/or medicinal response in a cell culture, tissue system, subject, animal, or human that is being sought by a researcher, veterinarian, clinician, or physician, which includes alleviation of the symptoms of the disease, condition, or disorder being treated.

The term “composition” is intended to encompass a product comprising the specified ingredients in the specified amounts, as well as any product that results, directly or indirectly, from combinations of the specified ingredients in the specified amounts.

Provided herein is a tissue-derived implant comprising: a plurality of decellularized extracellular cartilage particles, wherein each of the plurality of cartilage extracellular particles comprise one or more magnetic particles and MSCs.

The plurality of decellularized extracellular cartilage particles are derived from human, mouse, rat, dog, cat, rabbit, horse, pig, or nonhuman primate cartilage. In certain embodiments, the plurality of decellularized extracellular cartilage particles are derived from human or pig cartilage.

The plurality of decellularized extracellular cartilage particles can have an average diameter of 50-400 μm, 100-400 μm, 150-400 μm, 200-400 μm, 250-400 μm, 300-400 μm, 350-400 μm, 100-350 μm, 100-300 μm, 100-250 μm, 100-200 μm, 100-150 μm, 150-350 μm, 200-300 μm, 250-300 μm, 200-250 μm, or 176-300 μm. In certain embodiments, the plurality of decellularized extracellular cartilage particles have an average diameter of about 268 μm or 238±62 μm.

The plurality of decellularized extracellular cartilage particles can be prepared according to any method known in the art. In certain embodiments, the plurality of decellularized extracellular cartilage particles are prepared by treatment with an acid, a base, a surfactant, such as Triton® X-100, sodium dodecyl sulfate (SDS), and 3-((3-cholamidopropyl) dimethylammonio)-1-propanesulfonate (CHAPS), tributyl phosphate, hypertonic solution, hypotonic solution, an enzyme, such as trypsin, pepsin, an endonuclease, and an exonuclease, ethylene glycol tetraacetic acid (EGTA), and ethylenediaminetetraacetic acid (EDTA), and/or by physical means, such as freezing, force, agitation, vacuum-assisted decellularization (VAD), and hydrostatic pressure, or combinations thereof.

The composition of the one or more magnetic particles is not particularly limited and the present disclosure contemplates any substantially non-toxic material. In certain embodiments, the one or more magnetic particles comprise Fe₂O₃, Fe₃04, FeNi, FePt, Fe, CoNi alloy, a mixture thereof, or any magnetic nanomaterials made from one or more of these magnetic particles.

The one or more magnetic particles can have an average size of 50-300 nm, 100-300 nm, 150-300 nm, 200-300 nm, 250-300 nm, 100-250 nm, 100-200 nm, 100-150 nm, 150-250 nm, or 175-225 nm. In certain embodiments, the one or more magnetic particles can have an average size of about 200 nm.

The concentration of the one or more magnetic particles on the plurality of decellularized extracellular cartilage can be sufficient for an applied magnetic field to induce movement of the tissue-derived implant and below levels which substantially impact the health of the MSCs. In certain embodiments, the one or more magnetic particles have an average coating concentration on the plurality of decellularized extracellular cartilage particles of 2-20‰, 2-18‰, 2-16‰, 2-14‰, 2-12‰, 2-10‰, 2-8‰, 2-6‰, 2-4‰, 4-20‰, 6-20‰, 8-20‰, 10-20‰, 12-20‰, 14-20‰, 16-20‰, 19-20‰, 4-10‰, 4-6‰, 6-8‰, 6-10‰, or 8-10% wt/wt based on the weight of the one or more magnetic particles and the wet weight of the decellularized extracellular cartilage particles. In certain embodiments, the one or more magnetic particles have an average coating concentration on the plurality of decellularized extracellular cartilage particles of about 8 to about 10% wt/wt based on the weight of the one or more magnetic particles and the wet weight of the decellularized extracellular cartilage particles.

In certain embodiments, the MSC is an umbilical cord mesenchymal stem cell, a fetal mesenchymal stem cell, an umbilical cord blood mesenchymal stem cell, a placental mesenchymal stem cell, an amniotic fluid mesenchymal stem cell, a bone marrow mesenchymal stem cell, or an adipose mesenchymal stem cell.

In certain embodiments, the MSC are derived from bone marrow, adipose tissue, muscle, periodontal tissue, or dental pulp. In certain embodiments, the MSC are human, mouse, rat, dog, cat, rabbit, horse, pig, or nonhuman primate. In certain embodiments, the MSCs are human MSC. In certain embodiments, the MSCs are human MSC derived from bone marrow. The MSCs can be of autologous or allogeneic origin.

The MSCs can be present on the surface of the plurality of decellularized extracellular cartilage particles at seeding density of 100-5,000, 100-4,000, 100-3,000, 100-2,500, 100-2,000, 100-1,500, 100-1,000, 100-500, 500-3,000, 1,000-3,000, 1,500-3,000, 2,000-3,000, 2,500-3,000, 500-2,500, 500-2,000, 500-1,500, 500-1,000, 1,000-3,000, 1,500-3,000, 2,000-3,000, 2,500-3,000 1,000-2,500, 1,500-2,500, 2,000-2,500, 2,000-2,400, 2,100-2,300, or 1,500-2,000 MSCs per mm² of the plurality of decellularized extracellular cartilage particles. In certain embodiments, the MSCs are present on the surface of the plurality of decellularized extracellular cartilage particles at seeding density of about 2,200 MSCs per mm².

In certain embodiments, the tissue-derived implant further comprises one or more bioactive factor selected from the group consisting of Activin A and Activin B, Inhibin A, Inhibin B, TGF-α, TGF-β1, TGF-β2, TGF-β3, BMP-2, BMP-3, BMP-4, BMP-5, BMP-6, BMP-7, BMP-8, BMP-9, BMP-10, BMP-11, BMP-12, BMP-13, BMP-15, BMP-16, BMP-17, BMP-18, BMP-19, BMP-20, GDF-1, GDF-2, GDF-3, GDF-4, GDF-5, GDF-6, GDF-7, GDF-8, GDF-9, GDF-10, GDF-11, GDF-12, GDF-13, GDF-14, GDF-15, CDMP-1, CDMP-2, LMP-1, LMP-2, LMP-3, IGFBP1, IGFBP2, IGFBP3, IGFBP4, IGFBP5, IGFBP6, and kartogenin.

The present disclosure also provides a method of treating a cartilage defect in a subject in need thereof, the method comprising: administering a therapeutically effective amount of the tissue-derived implant described herein to the subject.

The cartilage defect can be the result of an injury, surgery, infection, malignancy, developmental malformation, and degenerative diseases such as osteoarthritis. In certain embodiments, the cartilage defect is the result of osteochondritis dissecans, articular collapse secondary to osteonecrosis or subchondral insufficiency fracture, osteochondral impaction fracture, osteoarthritis, chronic joint rheumatism, deformity arthritis, scapulohumeral periarthritis, cartilage degradation, anterior cruciate ligament injury, or surgery.

The site of the cartilage defect can be at the femoral condyle, the humeral head, the talus, or the capitellum of the humerus.

In certain embodiments, the tissue-derived implant is administered by intra-articular injection.

In certain embodiments, the method further comprises exposing the tissue-derived implant to a magnetic field thereby directing the tissue derived implant to the cartilage defect after administering the tissue-derived implant to the subject, wherein directing the tissue-derived implant to the cartilage defect visualized by ultrasound-guided photoacoustic imagining.

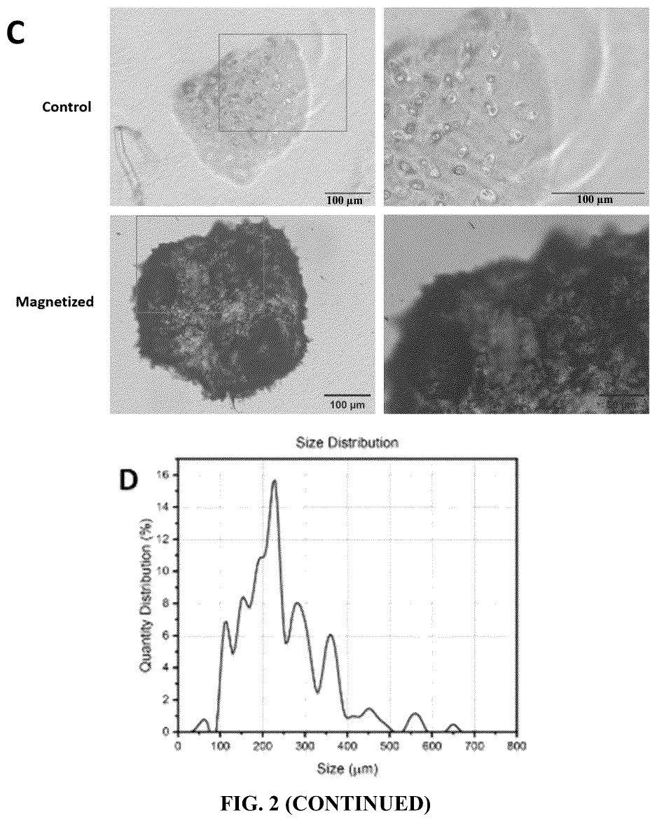

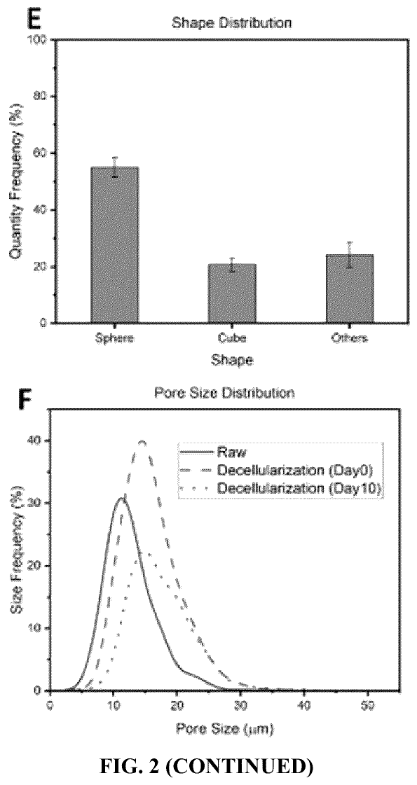

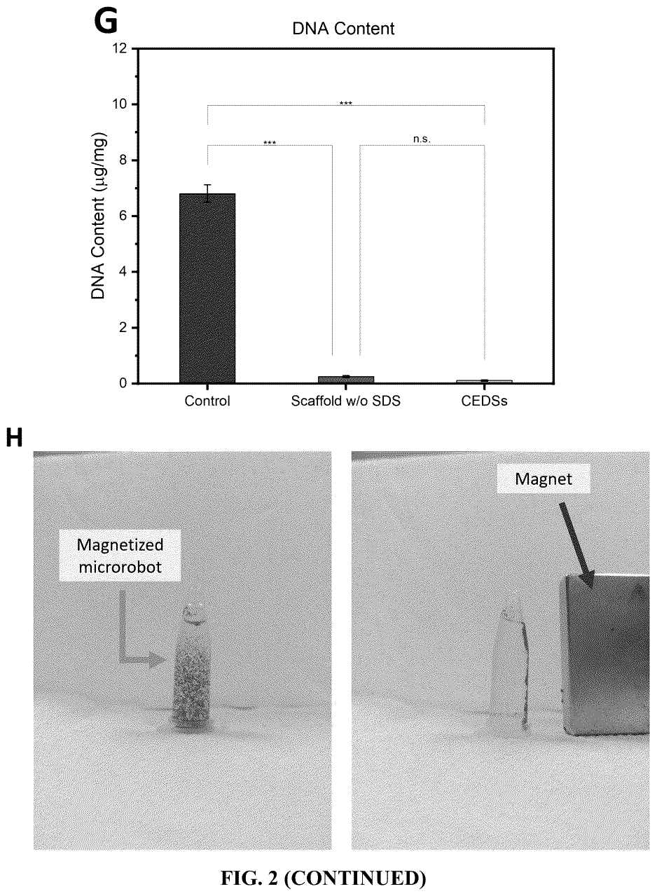

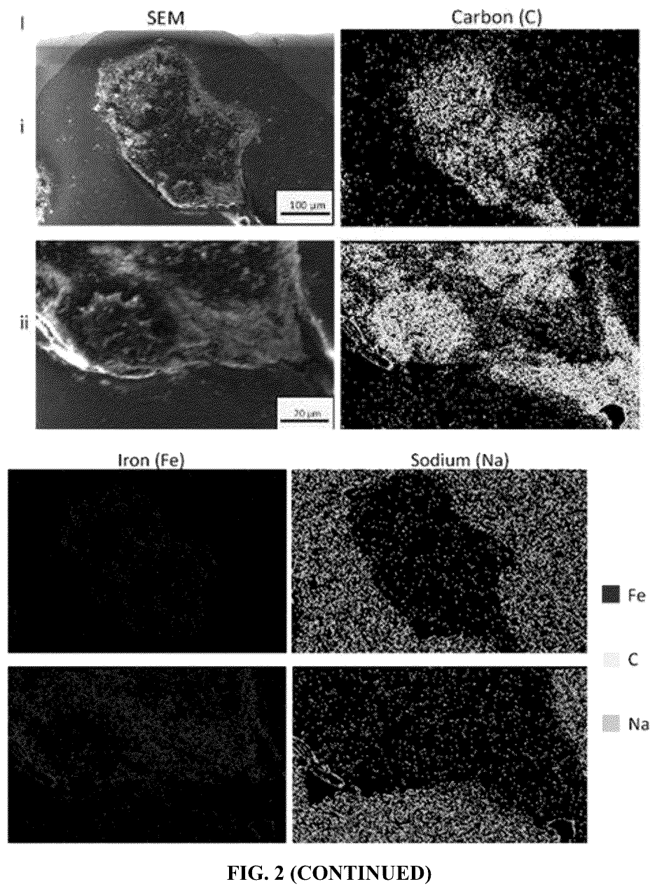

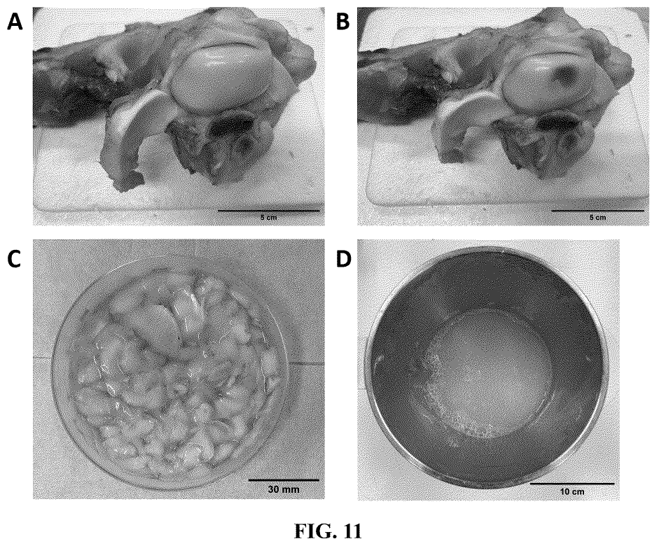

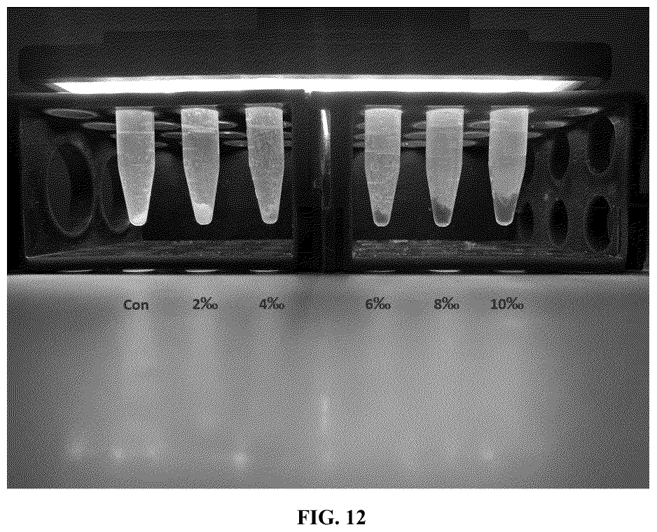

The microrobot described herein utilizes natural ECM-derived scaffolds obtained from fresh porcine knee joints, which possess porous structures that facilitate cell seeding. The initial steps involved obtaining the desired sizes and shapes of the natural scaffolds through cutting, grinding, and sieving of fresh porcine articular cartilages (FIGS. 2A and 11A-11D). Subsequently, the granular natural scaffolds were suspended in a storage buffer. FIG. 2B demonstrates the type II collagen fibers extended from the scaffolds, indicating natural collagen structures. Natural scaffolds with a wet size ranging from 100 μm to 300 μm were selected by filtering with nylon microporous membranes with 300 and 100 μm pores. The appearances of cartilage raw scaffolds and magnetized cartilage ECM-derived scaffolds (M-CEDSs) were observed under an inverted optical microscope where dark zones indicated magnetite deposition (FIGS. 12 and 2C). A wet size distribution indicated that the scaffolds' size, with a medium diameter of 238±62 μm, approached the size of commercial microcarriers, which benefited decellularization and magnetization ^{[9, 24, 41, 42]} (FIG. 2D). Spherical and cubic shapes dominated the population, which were used as models for simulation experiments (FIGS. 2E and 13). We then regarded the raw scaffolds as desirable for the downstream experiments.

The first significance of M-CEDSs is that the preservation of the hyaline cartilage structure promotes cellular events, including attachment, proliferation, and differentiation. Therefore, pore size distribution is a critical parameter for characterizing a microrobot's topography (FIG. 2F). Raw scaffolds and CEDSs in days 0 and 10 were investigated, showing distributions with average values of 11.45, 14.58, and 16.12 μm. These pore size distributions approached those in hyaline cartilage from humans (˜17.04 μm) and are thus useful in clinical applications and have been used in simulation studies ^{[43]}. Meanwhile, decellularization and degradation were quantified on the basis of size distribution. Animal tissues were used as raw materials; therefore, eliminating cells from a source is necessary to prevent biological pollution and donor impact. Raw scaffolds were treated with 1% sodium dodecyl sulfate (SDS), DNAse, and RNAse ^{[9, 44]}. The DNA content was quantified in raw cartilages, granular raw scaffolds, and CEDSs with a spectrophotometer to quantify the degree of decellularization. The test at 260 nm revealed a reduced DNA content compared with the control group and groups without SDS treatment, with DNA removal rates of 96.26% and 98.36%, respectively (FIG. 2G). Thus, thorough physical grinding processes significantly reduced the DNA content significantly. Overall, the reduction in the DNA content was aligned with other related decellularization studies ^{[9, 45]}.

To fabricate the M-CEDSs, the essential step is to magnetize the CEDSs while preserving the substructural integrity and intrinsic functionalities. The dip-coating magnetization technique was employed to coat Fe₃O₄ NPs onto decellularized scaffolds. Fe₃O₄ NPs stand out among materials because of their biocompatibility, innocuousness, and superparamagnetic features; their physiological effect on cell viability can be approximately neglected while magnetization performance can be maximized ^{[31]}. FIG. 2H reveals the magnetic properties of the M-CEDSs with a rectangular magnet. The coating of Fe₃O₄ NPs was successful during the dip-coating process; the rectangular magnet attracted M-CEDSs to one side of the centrifuge tube, overcoming gravity. For the convenience of future discussion, the coating concentration for M-CEDSs is defined as follows:

$$Coating Concentration ( {\%}_{0} ) = \frac{mass of magnetite nan oparticles}{wet mass of CED Ss to be coated} \times 100 \qquad ( 1 )$$

The magnetite nanoparticle distribution in M-CEDSs was mapped by using a scanning electron microscope equipped with an energy dispersive X-ray spectroscopy system (FIG. 2I). According to the results, M-CEDSs were identified clearly in carbon mapping where magnetite nanoparticles, identified as iron elements, were coated on the surfaces of M-CEDSs with a sparse distribution. Overall, magnetite nanoparticles were coated in the whole body of M-CEDSs; however, they did not occupy all the spaces of M-CEDSs, hindering cell attachment.

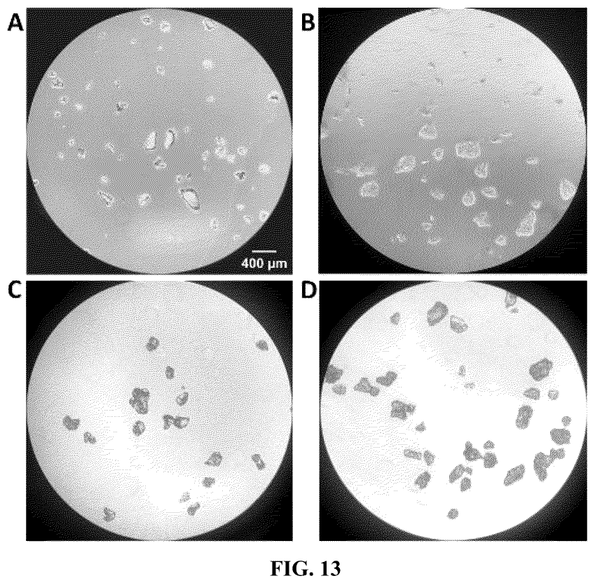

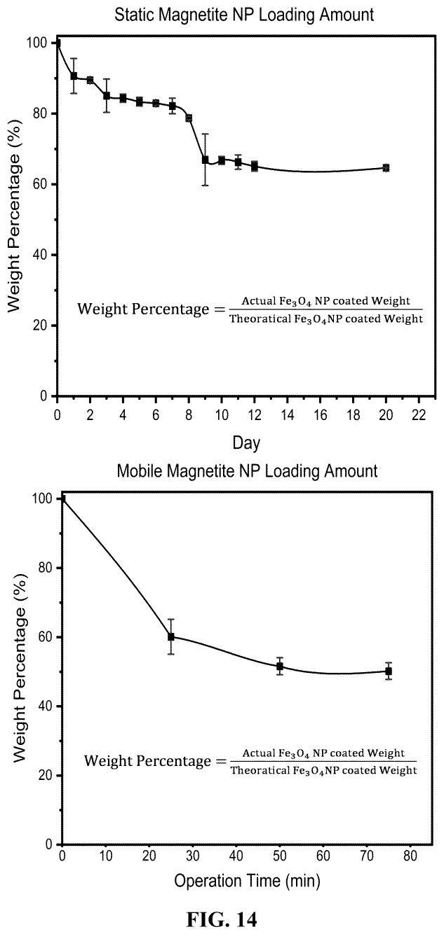

Finally, magnetite coating stability was measured. Spectrophotometry is a method widely applied to the nanoparticle concentration measurement ^{[46-48]}; therefore, after suspending M-CEDSs in stock solution, the amount of freely suspended magnetite nanoparticles can be measured from absorbance with a spectrophotometer. By deducing free magnetite nanoparticles from the total magnetite nanoparticle input, the practical loading amount of magnetite nanoparticles can be calculated. Our results indicated that approximately 90% of magnetite nanoparticles in the dip-coating solution will be practically coated to the scaffolds during the dip-coating process, then coated magnetite nanoparticles fell off the scaffold continuously and stabilized until 12 days after coating, reaching a value of 64% in a static environment; this stable value is approximately 50% in a mobile environment continuously driven by magnetic fields (FIG. 14). These findings demonstrated that the true coating concentration for a 10‰M-CEDSs was approximately 6.4‰. However, given that the practical value of a coating concentration is hard to control, we still used the nominal value as the coating concentration in this study.

Another significant advantage of using decellularized materials is the enhancement of mechanical properties and microrobot mobility in fluid environments. Mechanotransduction directly influences loaded cells, including the induction of MSC differentiation and the maintenance of the chondrocyte phenotype ^{[49-51]}. Notably, among all animal species, porcine articular cartilages exhibit the most similar collagen fiber arrangement structure to human articular cartilages ^{[52, 53]}. This arrangement resulted in a similar mechanical property between them, compared with microcarriers chemically manufactured, such as cytodex-3 ^{[54-17]}. Besides material properties, human articular hyaline cartilages and porcine cartilages have comparable pore sizes, whereas commercial microcarriers for cartilage engineering are solid or have disparate pore size distributions ^{[24, 58]}. Thus, the study used mechanical properties and porosity as key features in the comparison among human cartilage sphere, porcine-based M-CEDSs, and commercial microcarriers.

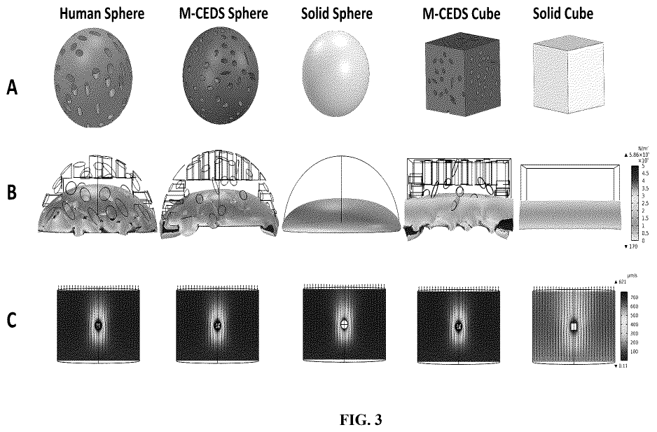

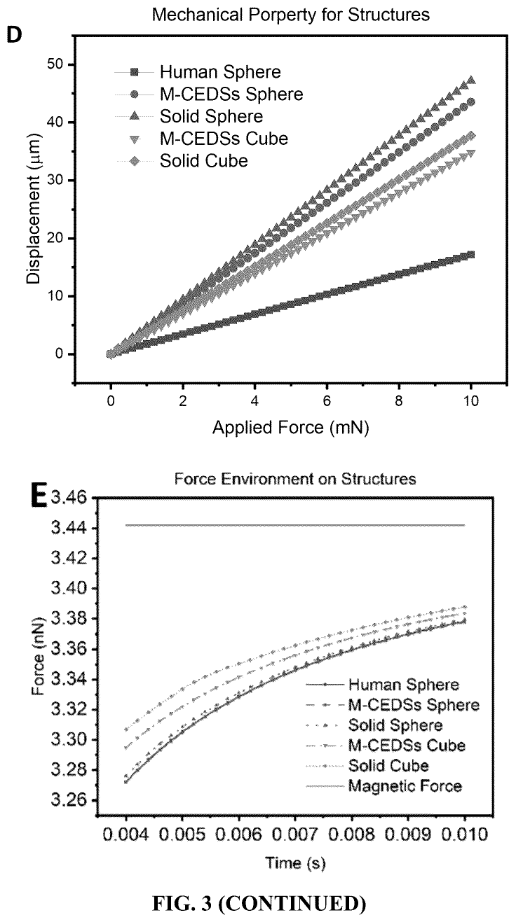

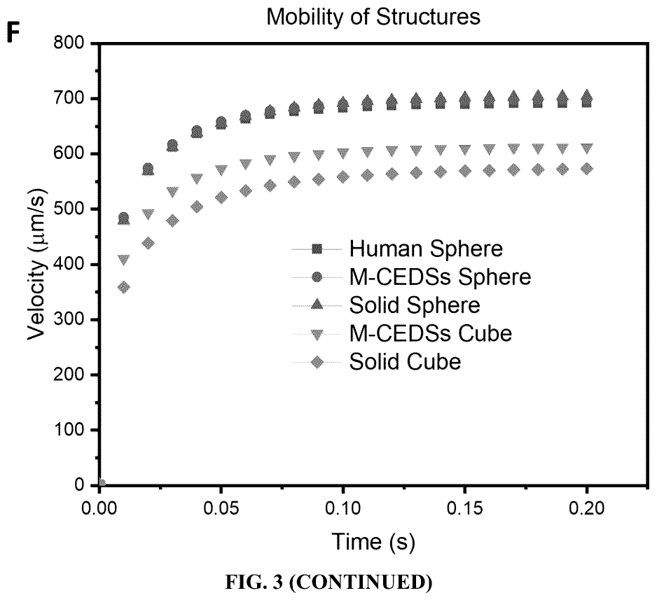

A numerical study using the finite element method was conducted on a human sphere, an M-CEDS sphere, a solid sphere, an M-CEDS cube, and a solid cube (FIG. 3A). For a succinct yet accurate simulation, all models were constructed with symmetrical shapes through engineering approximations. The human sphere is a model for human articular hyaline cartilages as a golden standard for microcarriers. According to our previous observation, the dominant shapes observed in the distribution were spherical and cubic; therefore, the M-CEDS sphere and M-CEDS cube were two models for the M-CEDSs, with diameter of 200 μm and side length of 161.2 μm, respectively. The commercially available cell microcarriers were modeled as solid spheres and solid cubes. The model parameters of all models were provided (FIG. 19). During compression tests, the stress distributions observed in the M-CEDS sphere and M-CEDS cube were similar to the stress distribution of the human sphere. This result suggested that the loaded cells in the M-CEDS models may have undergone a mechanotransduction process comparable to that in human articular hyaline cartilages. By contrast, the stress distributions observed in the solid models followed divergent paths (FIG. 3B). The fluidic velocity fields in the cross-sectional areas of all structures were visualized (FIG. 3C), showing smooth streamlines, except in the solid cube. In each shape, the slops of displacement curves of the M-CEDS groups were more closely aligned with the curve observed in humans, compared with the displacement curves of solid models, suggesting that M-CEDSs offer improved integration during implantation in future clinical trials (FIG. 3D). Overall, these results indicated that owing to the M-CEDSs' porcine-based source, M-CEDSs provide similar biomechanical environments with human cartilage. Other commercial microcarriers presented divergent environments, which hindered cellular activities, such as MSCs' directional differentiation and chondrocyte phenotype maintenance.

The driving process was then simulated. The volumes and driving forces of all structures were the same for a fair comparison, and the slight volume difference due to pores was neglected. Notably, the force environment exerted on the M-CEDS sphere was hardly different from that on the human sphere, correlating with the results in previous mechanical property tests (FIG. 3E). Finally, in the presence of the same driving magnetic force, increase in acceleration rate and enhanced steady speed represented increased magnetic driving capability. This phenomenon was consistent with the quantitative results where the M-CEDS cube had a much higher steady speed than the solid cube, whereas the porosity effect was minor in spherical groups (the human sphere, the M-CEDS sphere, and the solid sphere (FIG. 3F). The results of this study demonstrated that the M-CEDSs exhibited mechanical properties similar to those of human cartilage, and magnetic driving capacity higher than those of other commercially available microcarriers.

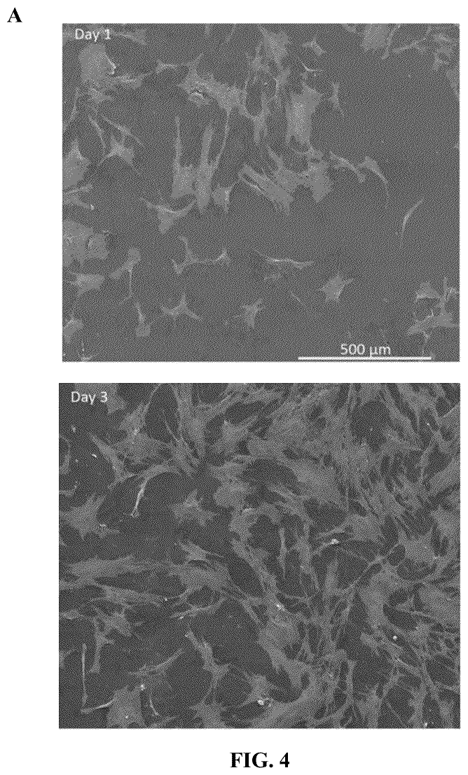

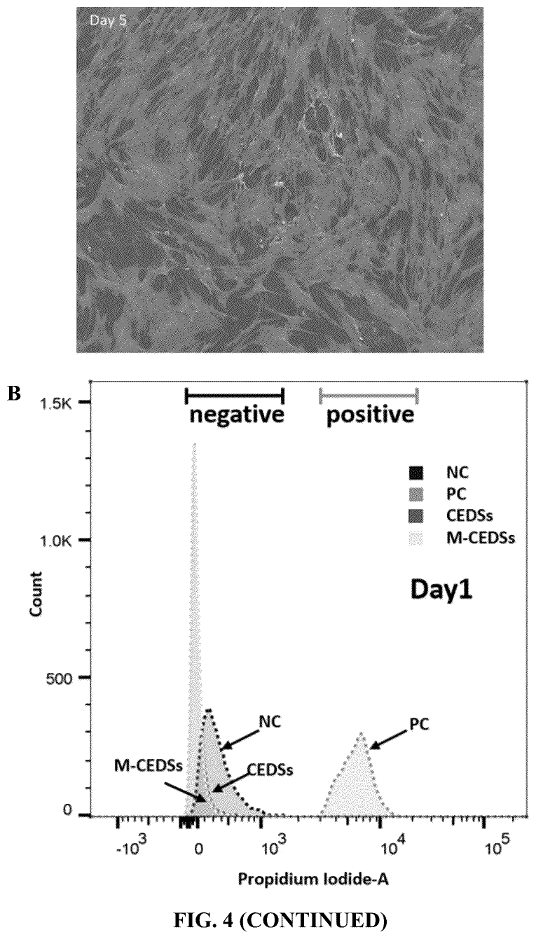

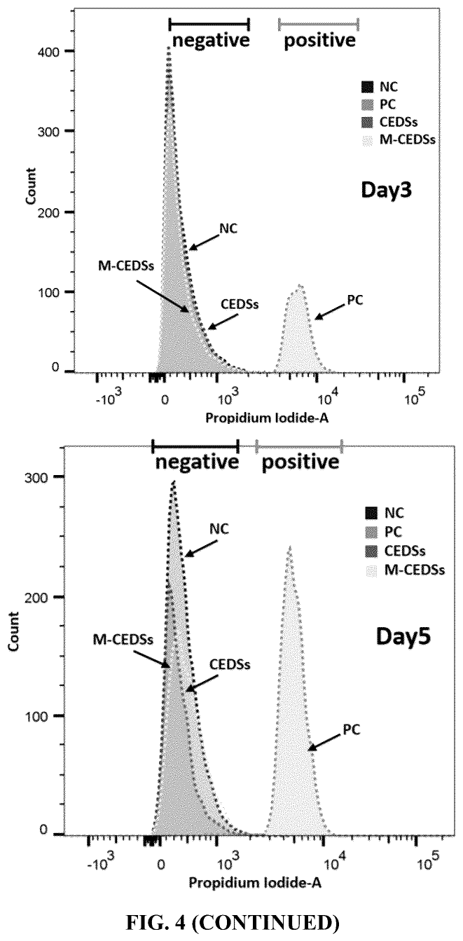

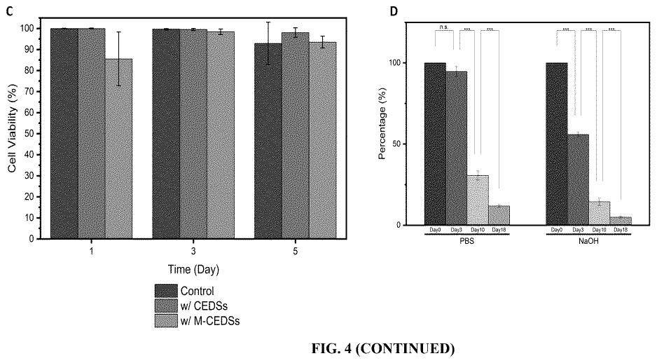

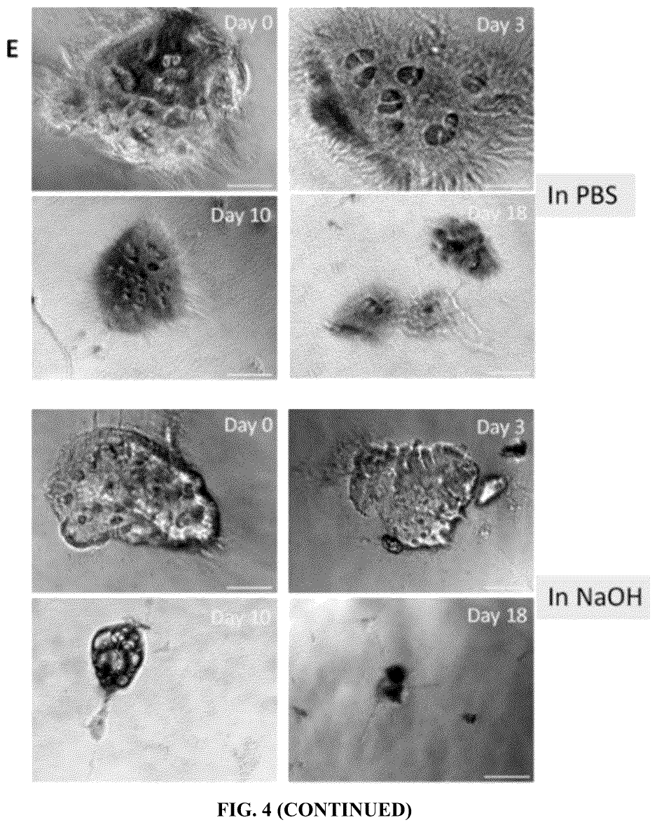

Cytotoxicity tests were performed on MSCs. First, cells cultured with Fe₃O₄ NPs were visualized by SEM on days 1, 3, and 5 (FIG. 4A). All cells exhibited good morphology and healthy growth densities. Second, the viability of cells cultured on CEDSs and M-CEDSs was quantified by using the propidium iodide (PI) histogram where cells cultured normally and treated with absolute ethanol were considered negative and positive controls, respectively. The cell viability of the M-CEDS groups was still acceptable after 5 days of culture (FIG. 4B). To further validate these results, we calculated the viability of the three groups separately: control, cells cultured with CEDSs, and cells cultured with M-CEDSs (FIG. 4C). All results showed that the CEDSs and M-CEDSs had low cytotoxicity to MSCs, which correlated with the cell seeding results.

Degradation tests were conducted for M-CEDSs in both PBS and NaOH environments until day 18 (FIG. 4D). M-CEDSs are mainly composed of collagen II; therefore, the collagen II degradation theory was used in this study ^{[59, 60]} Alkaline solutions are commonly used in collagen pretreatment ^{[61-63]} and extraction ^{[64-66]}, which degrade collagen telopeptides and intramolecular connectors involving proline and hydroxyproline; therefore, we used sodium hydroxide (NaOH) as a positive control to ensure that microrobots are fully degraded. Interestingly, we found that the degradation percentage reached over 69.37% within 10 days in PBS environments. This result can be attributed to the upstream SDS treatment during decellularization; SDS destabilizes the triple helical domain of collagen and influence elastin networks ^{[67-69]}. During decellularization, SDS degraded the collagen structure, increasing its solubility. Then, water molecules entered, facilitating hydrolysis, and the degradation of M-CEDSs in the downstream experiments. The observation was correlated with the quantitative results; initially, M-CEDSs had clear outlines and porous structures, but they became blurry on days 10 (PBS) and 3 (NaOH), presenting progressive degradation (FIG. 4E). The degradation results showed that M-CEDSs are degradable in natural environments within a justifiable period.

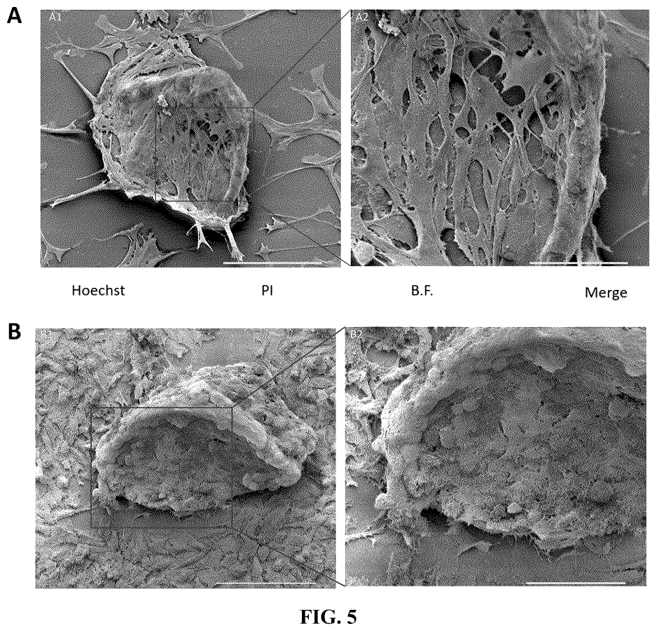

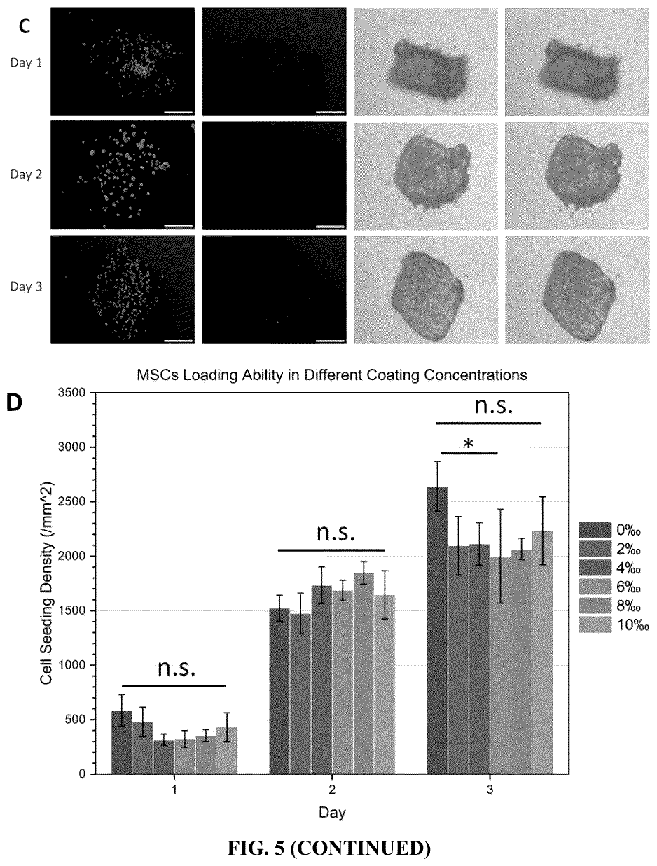

SEM images revealed the cell seeding ability of M-CEDSs with a coating concentration of 10‰, showing that human bone marrow MSCs and MC3T3-E1 fibroblasts were successfully seeded in the M-CEDSs after 3 days of culture at a seeding concentration of 1×10⁶ MSC or MC3T3-E1 cells/mL (FIGS. 5A1, A2, B1, and B2) on 2,500 M-CEDs particles/mL. Although this study focused on MSCs, MC3T3-E1 fibroblasts were used as positive controls because of their high proliferation rate and steady morphology ^{[32,70]}. As shown in FIGS. 5A and 5B, the loaded cell density was sufficient, and cell morphology was normal. Moreover, ECM was secreted around the cells in both groups, indicating normal growth on the microrobots.

Quantitative tests were conducted to analyze cell loading ability, which was defined as follows:

$$Cell loading ability = Cell number per scaffold area = \frac{1}{N} {\sum }_{i}^{N} \frac{{K}_{1}}{{A}_{i}} \qquad ( 2 )$$

where N is the M-CEDS number, K is the number of cells on the i-th M-CEDSs, and A is the projected area of the i-th M-CEDSs.

Living cells loaded on the M-CEDSs were highlighted through Hoechst 33258 and PI staining. As shown in FIG. 5C, during the 3 days of culture, the seeded MSCs on the M-CEDSs with coating concentrations of 0‰, 2‰, 4‰, 6‰, 8‰, and 10‰ proliferated constantly. Quantitative results were used to analyze the seeding density (FIG. 5D). M-CEDSs with average sizes loaded approximately 74 vial MSCs. Remarkably, statistical analysis reported that all coating concentrations had no significant difference in the same day, indicating that the Fe₃O₄ coating had marginal impact on cell growth. This result showed that high coating concentration can be adopted, that is, 10‰, to maximize delivery efficiency while maintaining high cell loading ability.

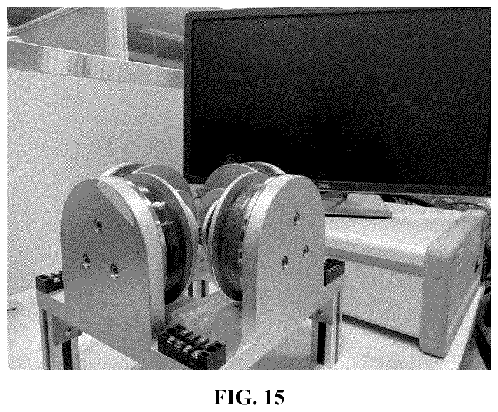

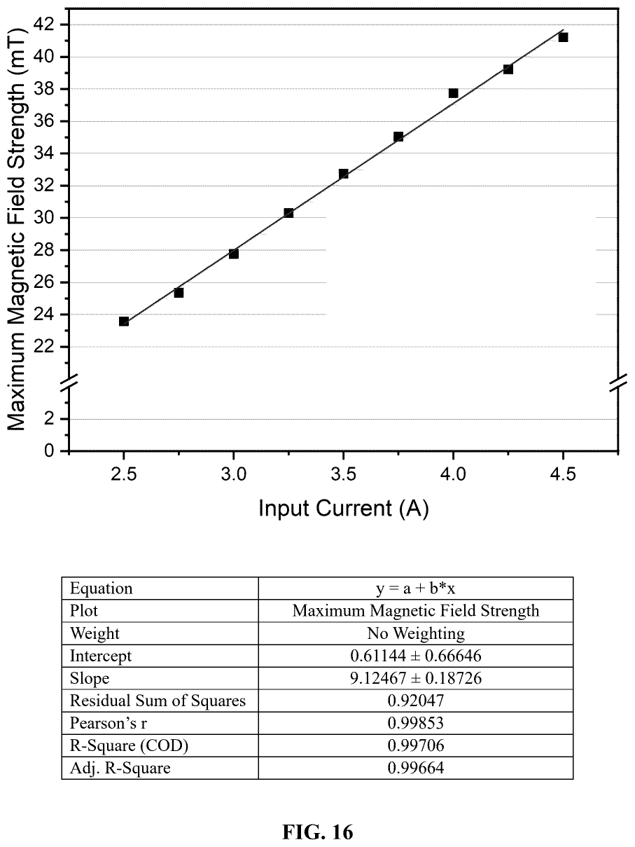

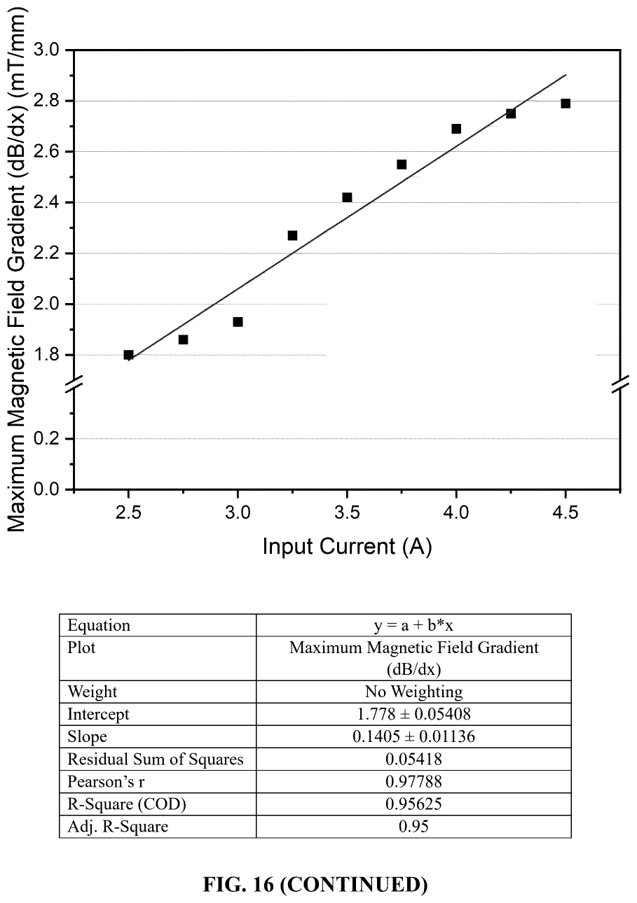

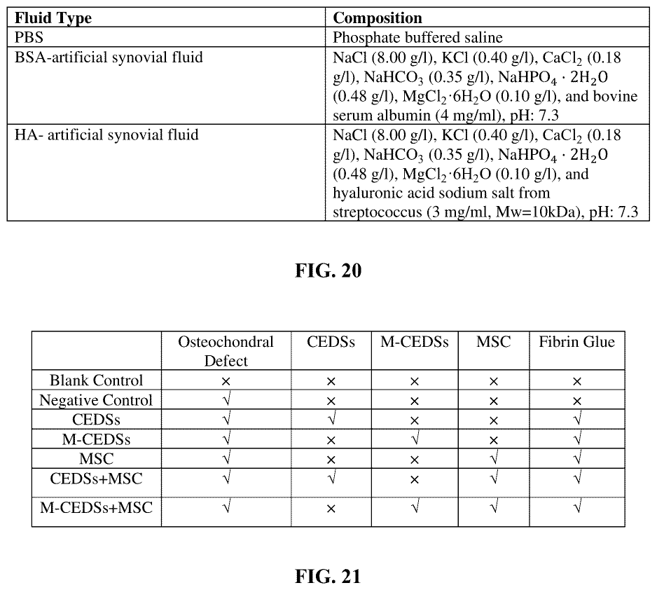

High mobility plays a central role in all cell-carrying microrobots. Here, the M-CEDSs were placed in different fluid environments, BSA-artificial synovial fluid, and HA-artificial synovial fluid (FIG. 20). The experiments were conducted in a self-constructed electromagnetic coil system reported by our laboratory to drive M-CEDSs ^{[39, 71, 72]} (FIG. 15). The electromagnetic properties of the self-constructed electromagnetic coils were tested, revealing the related magnetic field strength and gradient corresponding to the input currents (FIG. 16). The maximum working current of our coils was 20 A, which generated magnetic fields with magnetic field strength and magnetic field gradient exceeding 115 mT and 10 T/m, respectively. In the working environments in this study, magnetic field strength and magnetic field gradient did not exceed 37.11 mT and 2.34 T/m, respectively.

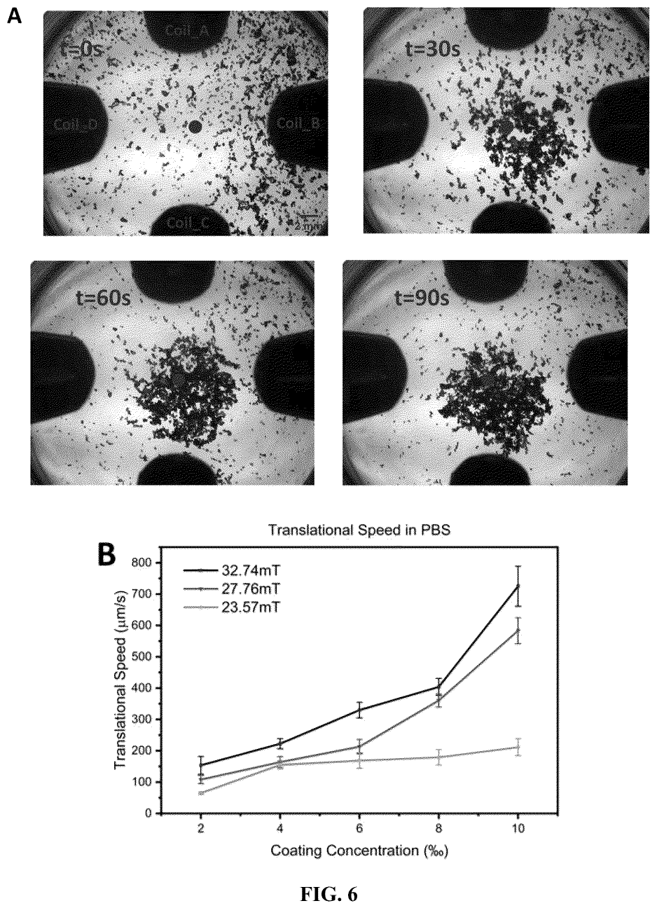

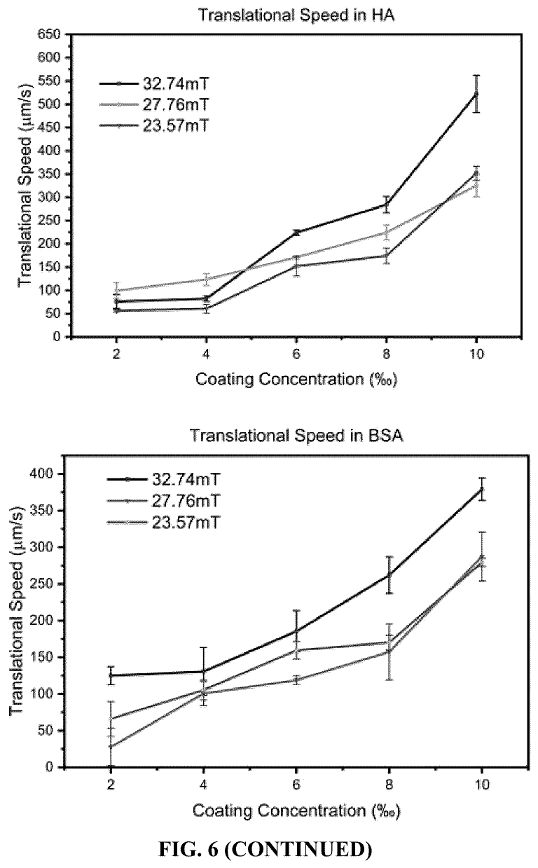

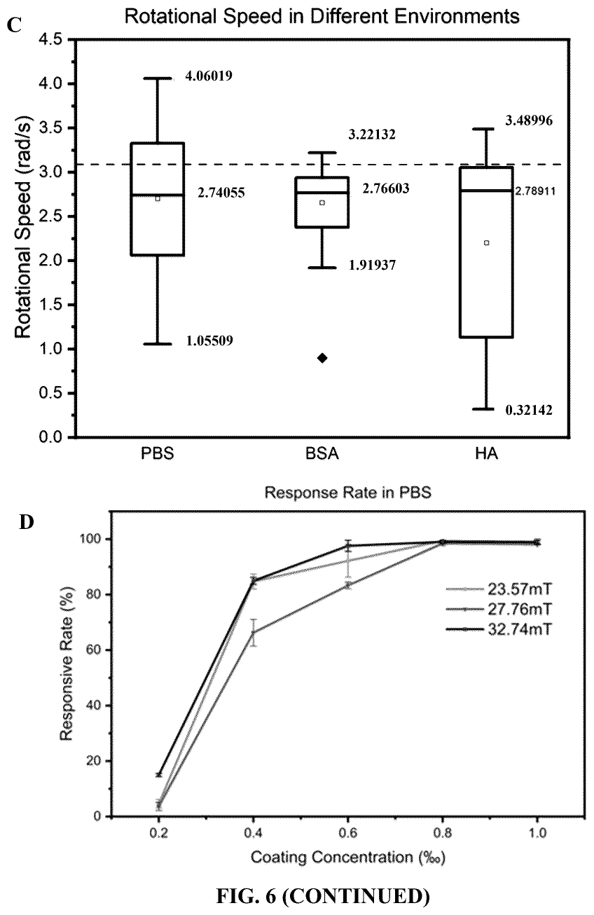

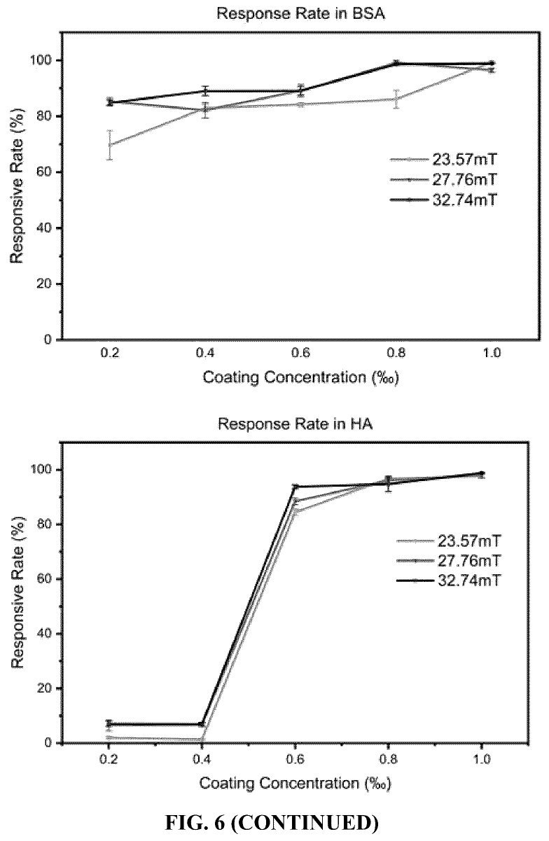

A typical aggregating process is depicted in FIG. 6A. The dispersive microrobots aggregated toward the geometric center of the system. Notably, the M-CEDSs' relatively large volumes increased inertia, decreasing aggregating frequency (˜0.5 Hz) and dispersing frequency (˜2 Hz), which were extremely low compared with those observed in other swarm microrobots ^{[71, 74]}. This phenomenon is hardly discussed because present microrobot's studies focus on uniformly small microrobots. Deep magnetization enhances mobility, but it presents concerns about mild cytotoxicity from coating materials, as shown in the previous section. As a novel study, the optimal coating concentration balancing cytotoxicity and mobility has not been addressed. This gap directed the following steps of this work. In this section, translational speed, rotational speed, and responsive rates were used for mobility quantification.

The motion of M-CEDSs was a composite motion from rotation driven by the RMFs and translocation driven by the magnetic gradients. They were calculated and analyzed separately. As shown in FIG. 6B, the M-CEDSs increased the translational speed significantly with increasing magnetic field strength (input currents). This effect was expected given that the magnetic field drives movements. In a magnetic field with a defined strength, M-CEDSs with a high coating concentration moved fast. M-CEDSs moved significantly faster in PBS than in the two artificial synovial fluids. The explanation behind this contrast was clear; the two artificial synovial fluids consisted of large molecules, increasing the viscosity and resistance inside the fluid. Therefore, in the environment with the largest magnetic strength, M-CEDSs in PBS had a 91.29% and a 38.84% speed increased compared with those in BSA-artificial and HA-artificial synovial fluids, respectively. However, the detailed explanation still needs in-depth studies given that the driving process involves complex factors, including viscosity, shape, and surface morphology, which were beyond the scope of this study. The rotational speeds were concentrated around the value of the external magnetic field rotating frequency (0.50 Hz, i.e., 3.14 rad/s) (FIG. 6C). However, in HA-artificial synovial fluid, M-CEDSs were not correlated with the critical value, indicating the high fluid friction in this environment.

However, the speed profile only revealed a partial situation during directional aggregation. In viscous fluids, such as HA-artificial synovial fluid, some M-CEDSs did not receive sufficient driving magnetic forces to overcome fluid friction. Therefore, quantifying the amount of M-CEDSs that can move in a magnetic field is crucial. Response rate is defined as the area ratio between M-CEDSs that rotated successfully and all M-CEDSs deposited (FIG. 6D). The results revealed that all groups showed increased responsive rates when they had high coating concentrations; however, the situation varied among different fluid environments. In the BSA-artificial synovial fluid, the coating concentrations of M-CEDSs did not act significantly, but they all had relatively higher response values than the other groups. This result can be explained by the bottom surface charge and friction modification inside the fluid. Low coating concentration diminished the response rates in the HA-artificial synovial fluid because of hyaluronic acid crosslinking networks. Thus, in all fluid environments, the coating concentration was more crucial than external magnetic field strength in terms of mobility.

Apart from aggregation, M-CEDSs' translocation motion along a preset rectangular path was recorded (FIG. 6Ei-6Eiv). All motions were driven directly by an open-loop magnetic actuation system. The microrobots moved rapidly and precisely, indicating reliable delivery in further applications.

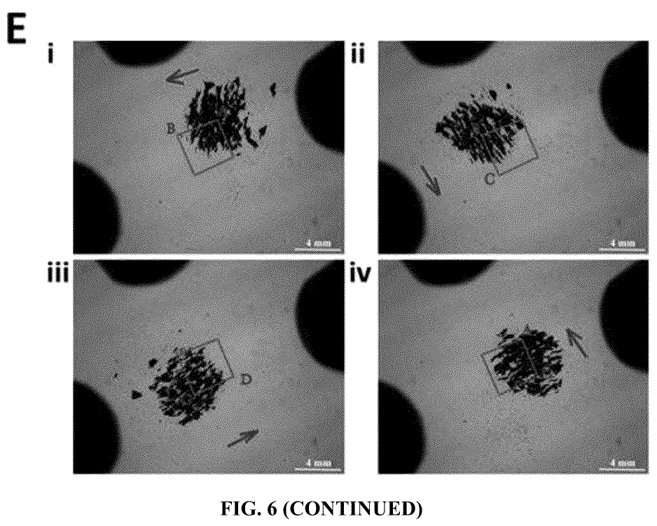

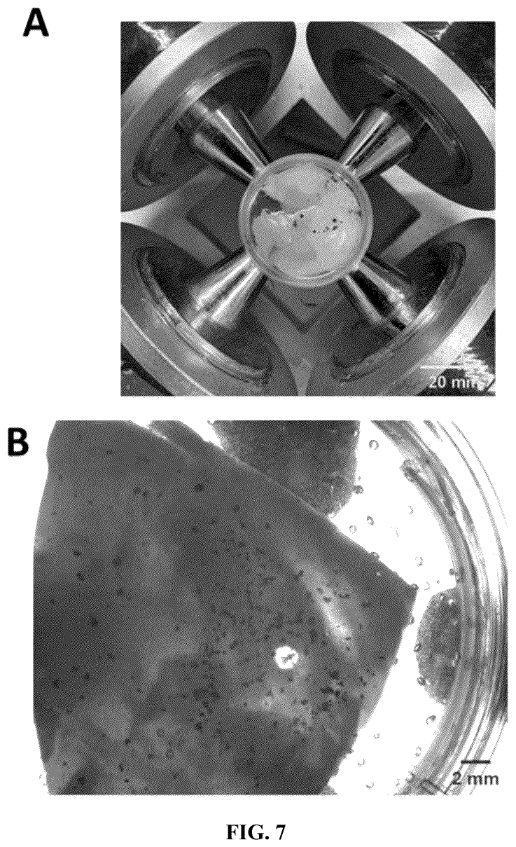

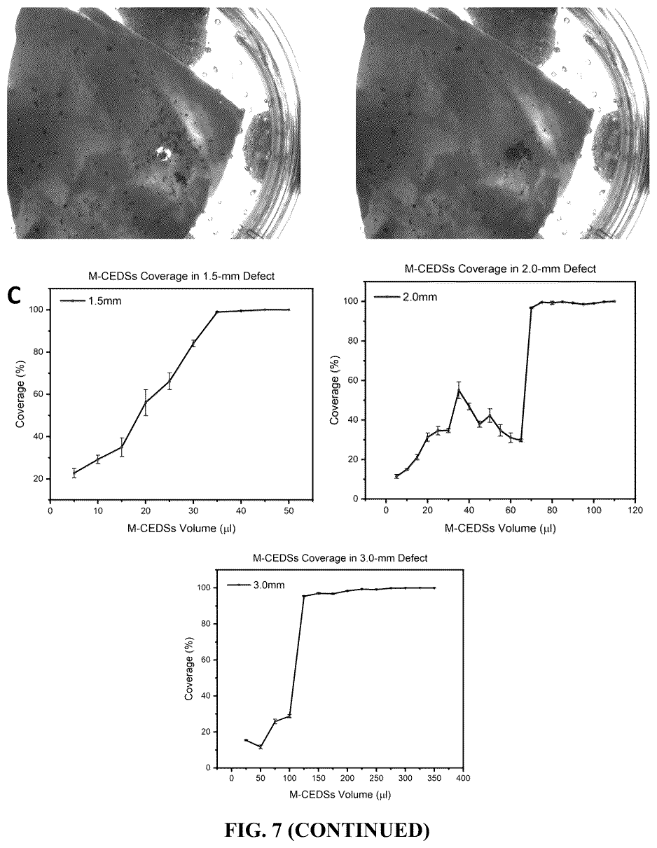

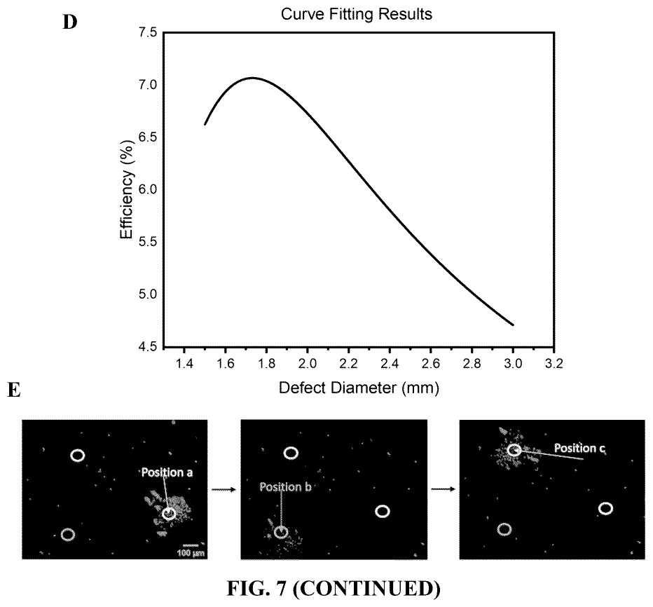

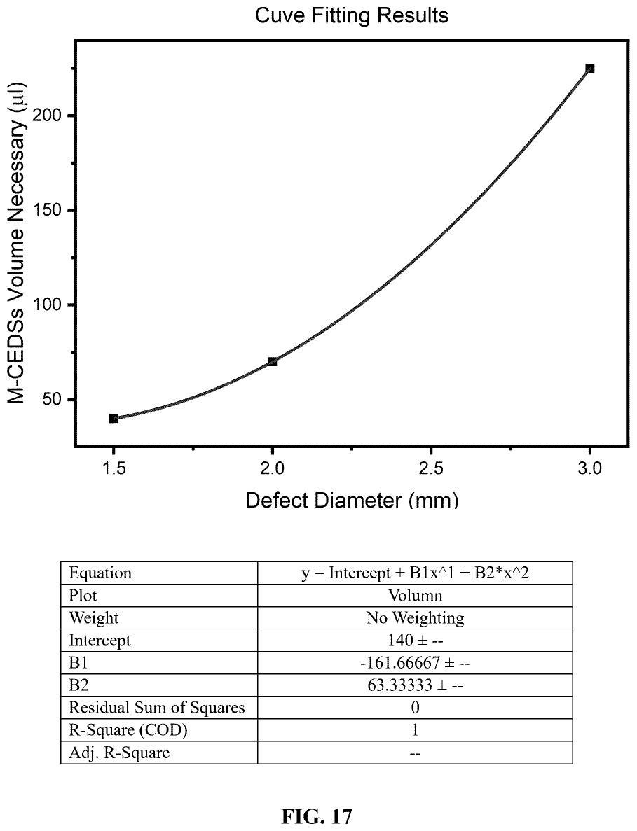

In vitro studies on real cartilage fragments revealed M-CEDSs' mobility and cell delivery ability (FIG. 7A). First, the filling model of M-CEDSs to chondral defects was established. Different amounts of M-CEDSs loaded with MSCs were aggregated to fill the chondral defects with diameters of 1.5, 2.0, and 3.0 mm (1.5 mm in depth; FIG. 7B). Coverage was defined as the area ratio between the covered area and the whole defect area. In a 1.5 mm defect, the coverage showed a proportional relationship with input volume; however, in the 2.0 and 3.0 mm defects, input volume did not have a clear pattern with coverage (FIG. 7C). This result was expected given that the heterogeneous shape randomizes a covered projection area in a small input volume. The necessary volume for M-CEDSs to fill a defect with a specific diameter can be predicted, along with the defect volume generated and filling efficiency (FIGS. 7D and 17). We can provide the defect-filling relation based on experimental results:

$${V}_{M - CEDSs} ( μL ) = 63.33 {D}^{2} - 161.67 D + 140 \qquad ( 3 )$$

where D is the diameter of defect size (unit: mm) with a depth of 1.5 mm.

Furthermore, cell delivery ability was depicted. The M-CEDSs loaded with MSCs were stained with Hoechst-33342, and magnetic fields were applied to drive composites toward a targeted site (FIG. 7E). Judging from the results, M-CEDSs loaded with MSCs can translocate to a different position rapidly and straightforwardly, showing cell delivery ability.

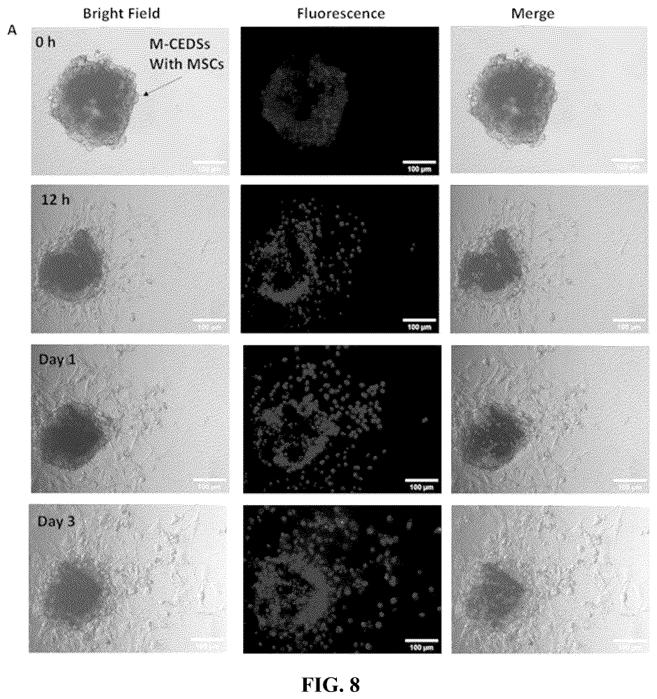

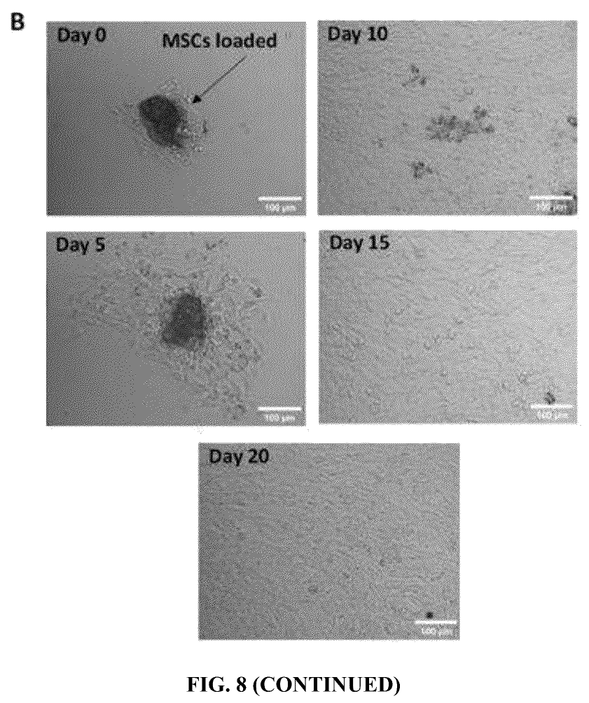

After translation to a desired site, only the successful release of seeding cells defined a suitable cell carrier. We supposed that seeded MSCs can be spontaneously released from the M-CEDSs to the surrounding environment because the cells are proliferating continuously, and cell aggregates continuously expand their area coverage. During seeding, scaffolds (M-CEDSs) are fully occupied by cells, and cells are squeezed out to surrounding environments; these phenomena have been demonstrated in our previous studies ^{[32, 33]}. For verification, we designed in vitro experiments for our microrobots. The status of MSC release was observed under a fluorescence microscope. After being seeded with MSCs overnight, M-CEDSs with a coating concentration of 10‰ were transported via magnetic fields carefully into a new position on a plastic surface for culture. FIG. 8A revealed the robust and gradual cell release from the M-CEDSs. Within 12 h after the microrobot reached a target site, the MSCs were released and attached to the environment stably. After 3 days of cultivation, the labeled MSCs proliferated onto the substrate, verifying the cell-delivery ability of M-CEDSs in vitro. Remarkably, the M-CEDSs were degraded after 20 days of cultivation (FIG. 8B), correlating with previous degradation results. The high growing densities and healthy morphology of the MSCs were observed after release. Therefore, the cell delivery from the microrobot in vitro on the 2D substrate was successful.

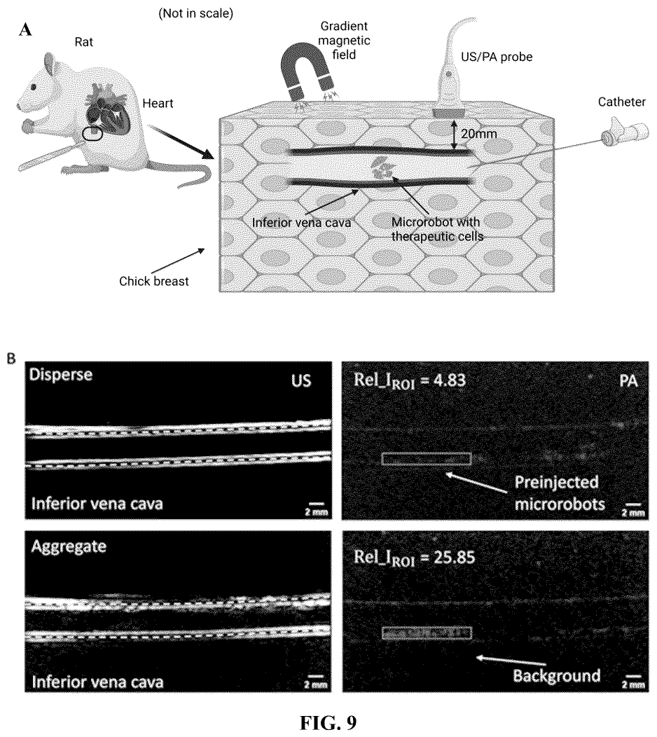

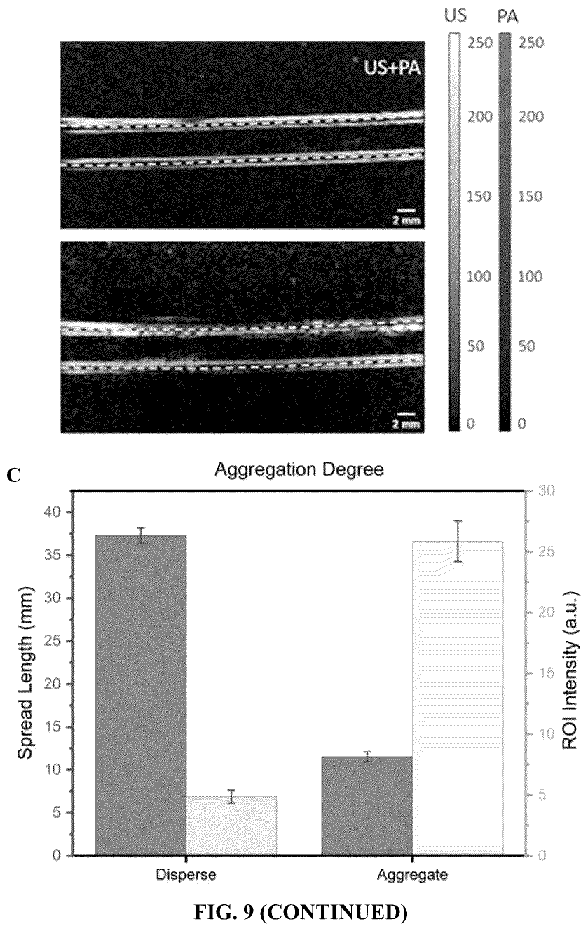

Owing to the lack of efficient and reliable imaging systems, conducting animal experiments for in vivo aggregation and cell delivery with our current imaging system was challenging; however, demonstrating that M-CEDSs have these abilities in vivo is necessary. To investigate the feasibility of aggregating and visualizing M-CEDSs, we used an ex vivo tissue phantom consisting of an inferior vena cava (IVC) dissected from a rat and a chicken breast (FIG. 9A). Given that a blood vessel is a complex environment, it can be used as a good model for testing the mobility of M-CEDSs in vivo, which has been used in other studies ^{[33, 75]}. The extracted IVC was buried horizontally inside a chicken breast at a depth of 20 mm. By soaking the phantom in a static fluidic environment, an M-CEDS cluster was injected and driven by the magnetic system. A flexible programmable ultrasonic system that can carry out both ultrasonic imaging and photoacoustic imaging tasks was used in visualizing the aggregation process. Given that the buried depth of the IVC was 20 mm, the deep-tissue aggregation ability of our magnetic system in vivo can be predicted and analyzed through this model.

Remarkably, M-CEDs had good contrast in deep PA imaging, and vessel walls were well distinguished in US imaging, providing a robust way in deep tissue imaging (FIG. 9B). The relative intensity of a region of interest reflected the number of microrobots, which increased from 4.83 to 25.85 a.u., showing an aggregation process (FIG. 9C). Meanwhile, the spread length indicated the occupied space of microrobots, which decreased from 37.27 mm to 11.50 mm, representing the aggregated state. All results showed that dispersed M-CEDSs aggregated inside the vessel driven by the external magnetic field. The aggregation process was rapid (≤1 s), showing a promising mobility of M-CEDSs in vivo. Unlike other works reported [³³, ⁷⁵], our work was carried out in a static fluidic environment; nevertheless, this environment is more analogous to the physiological situation inside the knee cavity ^{[76 77]}.

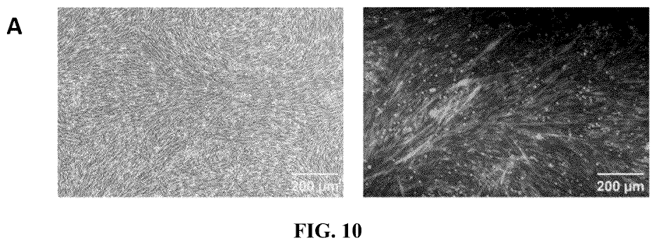

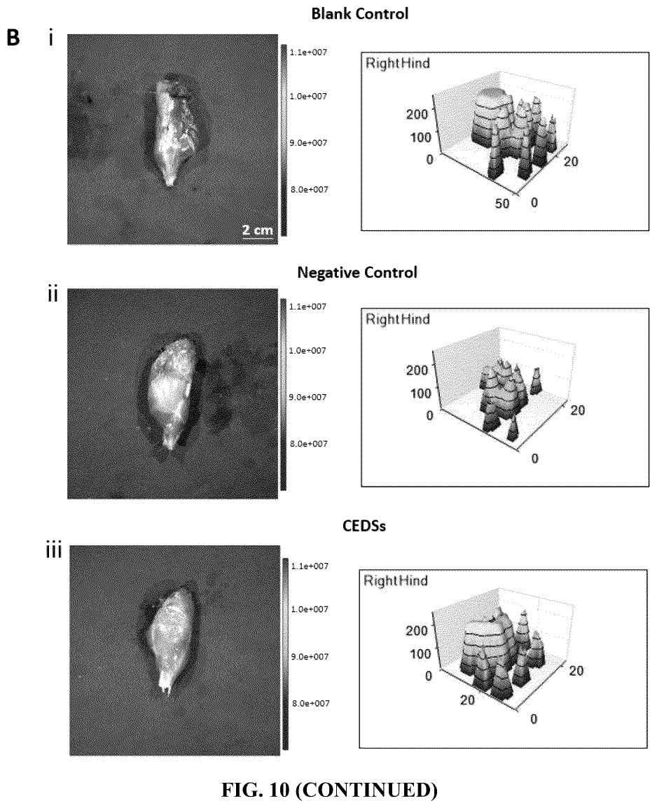

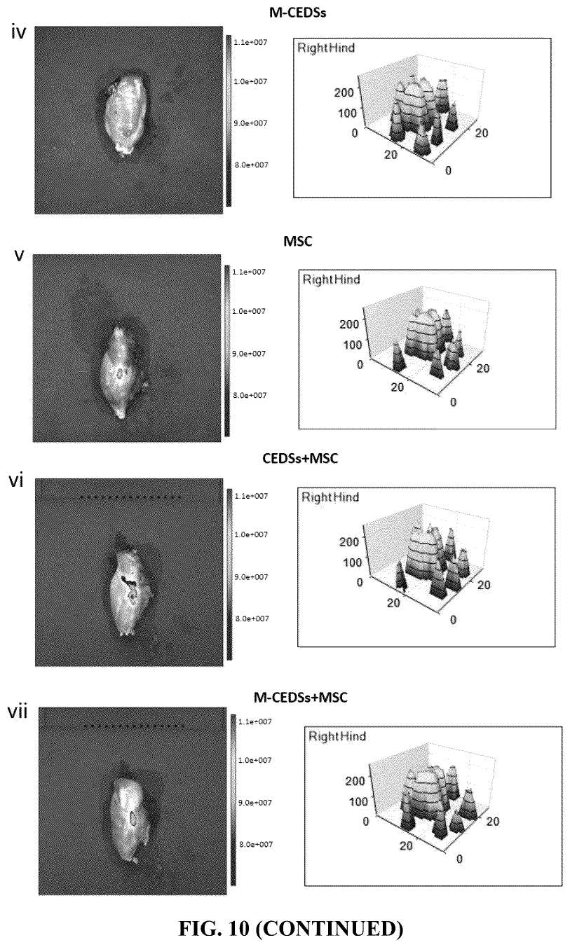

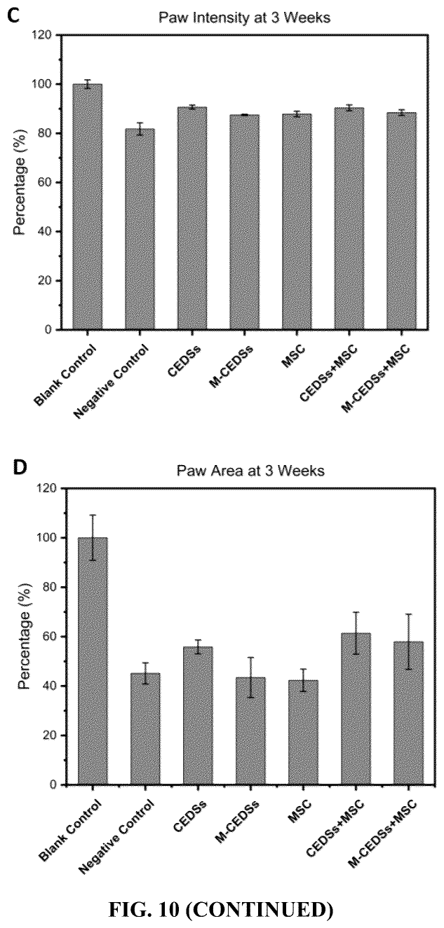

To investigate the in vivo therapeutical effects and the roles of implanted cells, we conducted gait analysis and fluorescent imaging 3 weeks after right hind knee surgery to analyze knee joint function (FIG. 10). In this experiment, seven groups were designed: blank control, negative control, CEDSs, M-CEDSs, MSC, CEDSs+MSC, and M-CEDSs+MSC groups. The details are provided in the experimental section (FIG. 21). In brief, the blank control group was composed of rats without surgery; the negative control group was composed of rats with osteochondral defects but without treatments; and the other groups were labelled according to the implants injected after osteochondral defects were created. Prior to the injection, rat BMSCs were labelled with a green fluorescent protein via the GFP lentivirus infection (FIG. 10A).

FIG. 10B shows the fluorescence signals of GFP-positive cells in cartilage defect (left) and 3D footprint intensity visualization (right) at 3 weeks in each group. Since fluorescence signals only appeared in groups treated with cell implantation, the results indicated that GFP-positive cells were delivered and remained in the repaired area 3 weeks after surgery. Meanwhile, the 3D footprint intensity visualization indicated that the M-CEDSs+MSC, CEDSs+MSC, and CEDS groups had closer intensity mapping than the blank control group. Paw mean intensity and paw area are two indexes to evaluate disability induced by the model in a CatWalk test, which has been used in movement-evoked pain studies ^{[78, 79]}. In these studies, a higher variance in paw mean intensity and paw area compared with control groups usually means a higher degree of movement-evoked pain. Given that the pain in our study was caused by knee joint surgery, these indexes quantified the knee joint function variation.

Quantitative results revealed that all groups experienced a decline in paw mean intensity, which was due to created cartilage defects (FIG. 10C). The paw mean intensity difference in experiment groups was not significant, which can be explained by both reduced contact force and paw contact area after surgery. The difference in knee joint function was more significant in FIG. 10D, where the paw area of the M-CEDSs+MSC group was 15.6% greater than that of the MSC group, indicating that M-CEDSs can promote cell activity. All groups showed knee joint function recovery compared with the negative control group, and our experimental group (M-CEDSs+MSC) resulted in a knee joint function recovery in both paw mean intensity (from 81.77% to 88.42%) and paw area (from 45.12% to 57.89%) at 3 weeks. This recovery value was higher than both MSC group and M-CEDSs group, showing that functional cells and proper microenvironments are critical in knee joint function recovery. The knee joints malfunctioned because of cartilage defects generated by surgery. Thus, we attributed the recovery of knee joint function to cartilage regeneration owing to implantation, MSC-seeded M-CEDSs. The results showed that cells were successfully delivered and remained at the defect sites, recovering the knee joint function where M-CEDSs could promote this activity.

In this study, fresh porcine articular cartilage was subjected to physical pulverization and chemical decellularization. Decellularized materials were selected to meet the requirements of biochemical environments, mechanical strength and magnetic actuation. The decellularization protocol was shown to result in a high decellularization degree. Cell culture assays confirmed no significant toxicity in either CEDSs or M-CEDSs. With the use of US/PA imaging, M-CEDSs could be visualized with a penetration depth of 2 cm where the aggregation was achieved with our self-constructed coil in an ex vivo tissue sample. The cells loaded in the microrobots could be spontaneously released to the cartilage defects in an animal model, resulting in an in vivo therapeutical effect. All the results demonstrated the feasibility of using the proposed microrobots for seeding and delivering functional cells to the desired site and proved the therapeutical effects of M-CEDSs for cartilage regeneration.

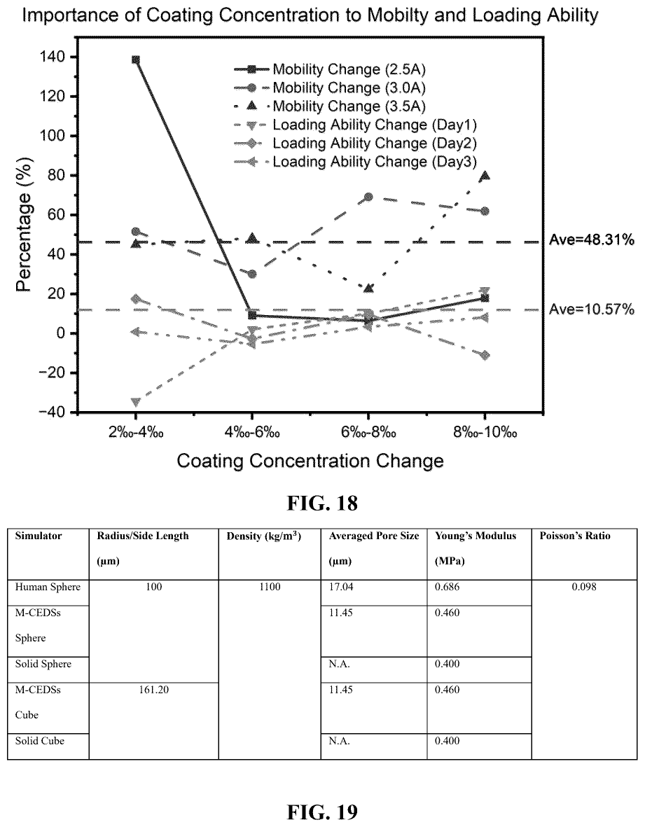

Magnetization is one of the originalities of this study compared with other parallel studies. The dip coating technique utilized for magnetization is easy and effective, and the mechanism behind this has been discussed in prior literature ^{[67, 80]}. However, to our best knowledge, there is no discussion about the magnetization protocol for decellularized materials. Additionally, due to the irregularity and relatively large scale, the aggregation and dispersion phenomenon of this type of microrobots has not been investigated. This paper found that the low aggregating and dispersing frequencies are suitable for M-CEDSs, which might be extended to the field of large-scale microrobots. This study conducted statistical analysis and found that the coating concentrations of M-CEDSs significantly affected mobility, but its negative effect on cell loading ability was minimal (FIG. 18). The average change resulting from every coating concentration increase was 48.31% in mobility but only 10.57% in cell loading groups. According to these results, a higher coating concentration only had a marginally negative effect on cell growth but a dominant positive effect on microrobot's mobility. Therefore, the maximum coating concentration of 10‰ was recommended to maintain high mobility while securing cell loading ability.

In terms of the response rate, M-CEDSs exhibited unexpected responsiveness inside BAS-artificial synovial fluid, which was beyond the initial assumptions of this study. This finding presents exciting opportunities for future investigations aimed at improving M-CEDS delivery efficiency in fluid environments. However, the HA-artificial synovial fluid posed a significant challenge to M-CEDSs' movement. Nevertheless, as HA-based fluids are widely used to alleviate symptoms of OA, enhancing the mobility of M-CEDSs in HA-based synovial fluid remains a valuable pursuit.

In this study, a defect-filling model was developed through in vitro experiments, and an empirical formula was derived by analyzing the volume-diameter relation. This formula provided a quantitative estimation of the input of M-CEDSs for future in vivo filling processes. However, the movement of M-CEDSs on the surface of the fresh cartilage fragments presented challenges, mainly due to the high electrostatic attraction between M-CEDSs and the cartilage surface. To overcome this challenge, one can increase the driving force with a strong magnetic field or perform surfactant deposit or surface polishing. Given the difficulties in increasing the strength of electromagnetic fields, we recommend adopting surface engineering as an essential step in future studies.

To further improve this work, the differentiation characteristics of MSCs should be investigated. Although some studies reported the facilitation of natural scaffolds in the induction of MSC differentiation, the influence of Fe₃O₄ NPs has not been discussed ^{[9, 44]}. Additionally, large animal models are required because mechanical environments considerably change with increasing body weight and the cartilage regeneration models vary.

Raw scaffold fabrication: Fresh porcine articular cartilage was cut with scalpels into fragments in a sterilized environment. The fragments were cleaned thoroughly with PBS and pulverized physically into raw scaffolds with an electrical grinder (DJ17A-D150; Jiuyang, Jinan, China). The raw scaffolds were filtered through a series of nylon microporous membranes with 300 and 100 μm pores sequentially. The fragments that penetrated the first sieve but were blocked by the second one had a diameter of 100-300 μm. The obtained raw scaffolds were suspended in PBS and treated rapidly to prevent protein degradation (FIG. 11).

Decellularization: The granular raw scaffold suspension was incubated with 1% SDS (25 mL/g cartilage) for 15 h on a shaker to maintain sufficient structural and biochemical components in the cartilage ECM, thereby minimize ultrastructure disruption to the native microenvironment ^{[9,68,69,81-84]}. All reagents were purchased from Sigma-Aldrich (Poole, the UK) unless otherwise specified. Furthermore, decellularized raw scaffolds were treated with 5 mL of deoxyribonuclease I (50 U/mL) and ribonuclease A (RNAse A; 1 U/mL) at room temperature for 4 h. After decellularization and 3% hydrogen peroxide sterilization for 30 min, the raw scaffolds were fabricated into cartilage ECM-derived scaffolds (CEDSs), which were rinsed at least 10 times with PBS to remove excess chemical reagents thoroughly.

Dip-coating: The CEDSs were magnetized via the dip coating technique by using an Fe₃O₄ NP solution (200 nm size, 25 mg/mL suspended in deionized water, Chemicell GmbH) ^{[33, 80, 85-89]}. To ensure that the NPs had a uniform dispersion, we treated the NP solution with ultrasound for 20 min before coating. The CEDSs were placed inside 50 mL centrifuge tubes and weighed. The dispersed magnetite solution was added slowly with a preset precise coating concentration, that is, wet weight ratio (1). The coating process proceeded for 15 h at room temperature under aseptic conditions. Finally, the M-CEDSs were rinsed at least 10 times with PBS to remove excess Fe₃O₄ NPs. The suspension was stored at 4° C. for further use (FIG. 12). The size and shape distributions of the CEDSs and M-CEDSs under wet and dry conditions were recorded under an inverted optical microscope (FIG. 13).

Magnetite nanoparticle loading amount: Before the magnetite nanoparticles were coated, the wet weight of the CEDSs were measured, and the amount of magnetite nanoparticles to be coated was calculated according to a specific coating concentration. After the 15-hr dip coating, the M-CEDSs were mixed thoroughly with the coating solution and left to stand at room temperature for 2 min for density separation. Finally, the upper layer solution was sampled, which was used in the static magnetite nanoparticle loading amount measurement. The same operation was carried out for 21 days. The mobile condition was driving the M-CEDSs in a rotating magnetic field continuously for 75 min, where the solution was sampled the in mobile magnetite nanoparticle loading amount measurement. Samples from different time points and groups were placed in an MD SpectraMAX M5e microplate reader for absorbance measurement at 645 nm. After calibrating with standard magnetite nanoparticle solutions, the concentration of magnetite nanoparticles in the sample, which was regarded as the amount of detached magnetite nanoparticles, was computed.

DNA quantification: Fresh porcine cartilage fragments, granular natural scaffolds, and CEDSs were prepared for comparison to examine the decellularization degree. The control group was the fresh porcine articular cartilage without any chemical treatment. The granularized scaffolds were obtained by grinding and sieving fresh cartilage without a chemical decellularization process, whereas the experimental groups were CEDSs. Three groups were all immersed in PBS and thawed at room temperature in a water bath at 37° C., vortexed, and centrifuged at 200 g for 5 min to obtain sufficient DNA remnants. RNAse A (1 U/mL) was added to both groups to eliminate the RNA content. A spectrophotometer (Biodrop Ltd., Cambridge, the UK) inspected the obtained DNA solution at 260 nm ^{[90]}. The experiment was conducted with three trials repeatedly.

Cytotoxicity: MSCs were obtained and adjusted to a density of 1×10⁶ vial cells/mL. Cells cultured with CEDSs and M-CEDSs were the two experimental groups, whereas cells cultured normally were used as the negative controls. Cells were incubated for 1, 3, and 5 days before harvesting and collecting. To construct a positive control, we washed the cells with 1 mL of PBS and 1 mL of absolute ethanol at −20° C. for 10 min for each 1.5 mL volume. All groups were stained with 1 mL of diluted PI (Thermo Fisher Scientific, catalog no. P1304MP) in a staining buffer (500 g/mL) at 100 μg/mL, and the cells were incubated for 15 min at 37° C. for flow cytometric analysis performed on a BD BioSciences flow cytometer (Becton, Dickinson and Company, Franklin Lakes, NJ, USA).

Biodegradability: M-CEDSs with 10‰ coating concentration were immersed in PBS and 10% NaOH environment at 37° C. In days 0, 3, 10, and 18, they were observed under an ECLIPSE Ts2R inverted microscope (Nikon, Tokyo, Japan) to investigate their morphology, which indicated their degree of degradation. Furthermore, the images were processed with Fiji for degradation percentage calculations.

Cell seeding on microrobots: Human bone marrow MSCs (ThermoFisher, A15652) were pre-cultured in Dulbecco's minimum essential medium (DMEM; Gibco, catalog no. 10567-014) supplemented with 10% fetal bovine serum (Gibco, catalog no. 10270-106), penicillin (100 U/mL), and streptomycin (100 U/mL; Invitrogen, catalog no. 15240-062) at 37° C. in a humidified atmosphere of 5% CO₂. The M-CEDSs with a coating concentration of 0‰, 2‰, 4‰, 6‰, 8‰, and 10‰ were sterilized with UV light and 3% hydrogen peroxide for 30 min to remove undesired microorganisms. In every 35 mm Petri dish, 300 μL of MSCs was added to the M-CEDSs at a concentration of 1×10⁶ vial cells/mL. A high seeding density was obtained by not using a sufficient volume of culture medium at this stage. After incubation at 37° C. for 1 h, 2 mL of DMEM was added to provide a sufficient growing condition with minimum nutrition. The microrobots seeded with MSCs were incubated overnight in a humidified incubator at 37° C. and 5% CO₂ for 2 days to stabilize cell attachment for further study. GFP-labeled MSCs were purchased directly (OriCell, RASMX-01101), and they were cultured and seeded on microrobots following the same protocol as normal cells.

Microrobot morphology: Granular raw scaffolds and CEDSs were placed on a cover glass in a Petri dish. After PBS was removed, they were fixed with 4% paraformaldehyde for 15 min and dehydrated with ethanol in an EM CPD300 critical point dryer (Leica, Wetzlar, Germany). We then sputtered them with gold in a QUORUM Q150TS dual-target sputtering system (Quorum Technologies, Lewes, the UK). The surface and pore architecture were observed with a FEI Quanta 250 environmental SEM (FEI, Hillsboro, USA). The morphology and porosity were then be analyzed directly. To reveal the carbon, iron, and sodium element distributions in M-CEDSs, we used a scanning electron microscope (JEOL, Tokyo, Japan) equipped with an energy dispersive X-ray system (Oxford Instruments, Abingdon, the UK).

Fluorescence imaging evaluation and quantification: The cell loading ability of the microrobots was evaluated on days 1, 2, and 3 according to the density of cell nuclei with an ECLIPSE Ts2R inverted microscope (Nikon, Tokyo, Japan). The cell-seeded microrobots were stained with Hoechst-33342 (Thermo Fisher Scientific, catalog no. 62249), and PI was used in counting living cells attached to the microrobots. Before staining, Hoechst-33342 and PI were diluted with PBS to final concentrations of 1 and 100 μg/mL, respectively. The original culture medium in the culture dish was replaced with 1.5 mL of Hoechst-33342 solution to cover the cells. After the MSC-loaded microrobots were incubated at 37° C. for 10 min, another 1.5 mL of PI was added, and the cells were incubated under the same condition for another 10 min. Finally, living cells attached to each microrobot was counted separately via Fiji or flow cytometry.

Mobility test: A self-constructed electromagnetic coil setup was applied to drive M-CEDSs on 2D substrates and a 3D environment, and perform aggregation and transportation tasks ^{[32, 39, 71]} (FIG. 15). To measure mobility, we placed sufficient microrobots with coating concentrations of 0‰, 2‰, 4‰, 6‰, 8‰, and 10‰ in 35 mm test Petri dishes covered with PBS. The substrate was placed on the working area of our electromagnetic platform. A rotation magnetic field was actuated to induce aggregation at a specific aggregation center at 0.5 Hz in the presence of varying magnetic field strength (23.57, 27.76, and 32.74 mT; input currents: 2.5, 3.0, and 3.5 A). The electromagnetic properties of this platform, including magnetic strength and gradient, were tested (FIG. 16). A rectangular movement was achieved by setting up different aggregation centers continuously with time along the preset paths [³⁹]. The mobility of the microrobots was examined in two types of synovial fluids, namely, BSA-artificial and HA-artificial synovial fluids, which simulated the real human knee environment's physical properties ^{[91, 92]} (FIG. 20).

Defect-filling model and delivery of cells from microrobots: Fresh cartilage fragments were produced in previous steps. Chondral defects (1.5, 2.0, and 3.0 mm in diameter; 1.5 mm in depth) were generated by using a dental drill. After fixing the cartilage within Petri dishes, we placed them in the electromagnetic coil system. MSCs were seeded in M-CEDSs with a coating concentration of 10‰, and the composite solution was adjusted to a density of 1002.21 mg/mL. The composites were carefully added to the cartilage fragments. Lastly, the RMFs were actuated, transporting M-CEDSs loaded with MSCs toward the defect sites.

In vitro cell release onto the substrate: To observe cell release, we isolated the M-CEDSs loaded with MSCs and placed them in a Petri dish. After being driven to the targeted site with RMFs, they were incubated at 37° C. in a humidified atmosphere of 5% CO₂. At 0, 12 h, and 1, 2, 3, 5, 10, 15, and 20 days, they were observed under the ECLIPSE Ts2R inverted microscope (Nikon, Tokyo, Japan) and cell release status was determined.

Imaging and magnetic manipulation of the microrobots in tissue phantoms: To observe microrobot aggregation inside tissues, an IVC was dissected from a rat. The extracted IVC was buried horizontally inside a fresh chicken breast sample with a depth of 20 mm. The tissue sample was immersed in an uncovered plastic container filled with water. After placing the container in our self-constructed electromagnetic coils, we used a Verasonic Vantage 128 system (Verasonics, Washington, USA) equipped with Dava 300 laser (Beamtech, Beijing, China) as the US/PA imaging system.

Rat femoral trochlear cartilage defect model and repair with GFP-MSC-loaded M-CEDSs: Forty-two healthy male Sprague-Dawley rats weighing 300-350 g were used according to the protocol approved by the Peking University Third Hospital Medical Science Research Ethics Committee (IRB00006761-M2021216). The animals were anesthetized with 3% sodium pentobarbital (40 mg/kg body weight, i.p.) and then an osteochondral defect was created with a sterile biopsy punch in the center of the trochlear groove of the right hind leg. The rats were randomly divided into seven groups according to the implants: the blank control group, in which no surgery operation was carried out; the negative control group, in which only an osteochondral defect was applied; the CEDSs group, in which CEDSs were implanted alone into the defects and fibrin glue was applied to the surface to secure the scaffolds; the M-CEDSs group, in which M-CEDSs were implanted alone into the defects and fibrin glue was applied to the surface to secure the scaffolds; the MSC group, in which GFP-MSCs were implanted into cartilage defects and fibrin glue was applied to the surface to secure the cells; the CEDSs+MSC group, in which the prepared composites of GFP-MSC-laden CEDSs were implanted into cartilage defects, and fibrin glue was applied to the surface to secure the scaffolds; the M-CEDSs+MSC group, in which the prepared composites of GFP-MSC-laden M-CEDSs were implanted into cartilage defects, and fibrin glue was applied to the surface to secure the scaffolds (FIG. 21). The rats were sacrificed by CO₂ asphyxiation 3 weeks after surgery.

Gait analysis: Gait analysis was conducted on walking rats (n=6) 3 weeks after surgery by using the catwalk method (CatWalk XT, Wageningen, The Netherlands). The mean footprint intensity (unit: a.u.) reflected the pressure exerted by the paw and paw area (unit: cm²) represented the contact area, which are the two common evaluation indexes for knee joint function. All data were measured from the experimental right hind legs.

Fluorescence imaging for knee joints in vivo: After the rats were sacrificed by C0₂ asphyxiation 3 weeks after surgery, experimental rat knee joints were taken and examined with Kodak In-Vivo Imaging System FX (Kodak, Tokyo, Japan) to track the GFPBMSCs in the repaired area 3 weeks after surgery, as described previously.

Statistical analysis: All data in this study is shown as mean±standard deviation. One-way ANOVA statistical analysis with post hoc comparisons was used in data analysis. p≤0.05 indicated statistical significance.

### REFERENCES

[1] D. J. Hunter, L. March, and M. Chew, “Osteoarthritis in 2020 and beyond: a Lancet Commission,” The Lancet, vol. 396, no. 10264, pp. 917-921, 2020, doi: 10.1016/s0140-6736(20)32230-3.
[2] D. J. H. C. H. K. A. ATHANASIOU, “Unlike bone, cartilage regeneration remains elusive,” SCIENCE, vol. 338, no. 6109, pp. 917-921, 16 Nov. 2012 2012.
[3] J. A. Buckwalter, C. Saltzman, and T. Brown, “The impact of osteoarthritis: implications for research,” Clinical Orthopaedics and Related Research®, vol. 427, pp. S6-S15, 2004.
[4] N. Arden and M. C. Nevitt, “Osteoarthritis: epidemiology,” Best practice & research Clinical rheumatology, vol. 20, no. 1, pp. 3-25, 2006.
[5] J. N. Katz, K. R. Arant, and R. F. Loeser, “Diagnosis and treatment of hip and knee osteoarthritis: a review,” Jama, vol. 325, no. 6, pp. 568-578, 2021.
[6] U. Noth, A. F. Steinert, and R. S. Tuan, “Technology insight: adult mesenchymal stem cells for osteoarthritis therapy,” Nature clinical practice Rheumatology, vol. 4, no. 7, pp. 371-380, 2008.
[7] H. Ohgushi et al., “Tissue engineered ceramic artificial joint—ex vivo osteogenic differentiation of patient mesenchymal cells on total ankle joints for treatment of osteoarthritis,” Biomaterials, vol. 26, no. 22, pp. 4654-4661, 2005.
[8] M. F. Pittenger et al., “Multilineage potential of adult human mesenchymal stem cells,” science, vol. 284, no. 5411, pp. 143-147, 1999.
[9] H. Yin et al., “Induction of mesenchymal stem cell chondrogenic differentiation and functional cartilage microtissue formation for in vivo cartilage regeneration by cartilage extracellular matrix-derived particles,” Acta Biomater, vol. 33, pp. 96-109, March 2016, doi: 10.1016/j.actbio.2016.01.024.
[10] Q. Yang et al., “A cartilage ECM-derived 3-D porous acellular matrix scaffold for in vivo cartilage tissue engineering with PKH26-labeled chondrogenic bone marrow-derived mesenchymal stem cells,” Biomaterials, vol. 29, no. 15, pp. 2378-2387, 2008.
[11] Y Li, H. Cheng, K. Cheung, D. Chan, and B. Chan, “Mesenchymal stem cell-collagen microspheres for articular cartilage repair: cell density and differentiation status,” Acta biomaterialia, vol. 10, no. 5, pp. 1919-1929, 2014.
[12] A. J. Steward and D. J. Kelly, “Mechanical regulation of mesenchymal stem cell differentiation,” Journal of anatomy, vol. 227, no. 6, pp. 717-731, 2015.
[13] D. Brahatheeswaran, Y Yasuhiko, M. Toru, and K. D. Sakthi, “Polymeric scaffolds in tissue engineering application: a review,”International Journal of Polymer Science, vol. 2011, no. 290602, pp. 1-19, 2011.
[14] L. Freed, G. Vunjak-Novakovic, and R. Langer, “Cultivation of cell-polymer cartilage implants in bioreactors,” Journal of cellular biochemistry, vol. 51, no. 3, pp. 257-264, 1993.
[15] D. Schop, F. Janssen, E. Borgart, J. De Bruijn, and R. van Dijkhuizen-Radersma, “Expansion of mesenchymal stem cells using a microcarrier-based cultivation system: growth and metabolism,” Journal of tissue engineering and regenerative medicine, vol. 2, no. 2-3, pp. 126-135, 2008.
[16] M. Mathieu, S. Vigier, M. Labour, C. Jorgensen, E. Belamie, and D. Nod1, “Induction of mesenchymal stem cell differentiation and cartilage formation by cross-linker-free collagen microspheres,” eCells and Materials Journal, vol. 28, pp. 82-97, 2014.
[17] Y Qi, Y Du, W. Li, X. Dai, T. Zhao, and W. Yan, “Cartilage repair using mesenchymal stem cell (MSC) sheet and MSCs-loaded bilayer PLGA scaffold in a rabbit model,” Knee Surg Sports Traumatol Arthrosc, vol. 22, no. 6, pp. 1424-33, June 2014, doi: 10.1007/s00167-012-2256-3.
[18] O. F. W. Gardner et al., “Asymmetrical seeding of MSCs into fibrin-poly(ester-urethane) scaffolds and its effect on mechanically induced chondrogenesis,” J Tissue Eng Regen Med, vol. 11, no. 10, pp. 2912-2921, October 2017, doi: 10.1002/term.2194.
[19] M. W. Pot, L. M. de Kroon, P. M. van der Kraan, T. H. van Kuppevelt, and W. F. Daamen, “Unidirectional BMP2-loaded collagen scaffolds induce chondrogenic differentiation,” Biomedical Materials, vol. 13, no. 1, p. 015007, 2017.
[20] B. Grigolo et al., “Osteoarthritis treated with mesenchymal stem cells on hyaluronan-based scaffold in rabbit,” Tissue Engineering Part C: Methods, vol. 15, no. 4, pp. 647-658, 2009.
[21] X. Xie, C. Zhang, and R. S. Tuan, “Biology of platelet-rich plasma and its clinical application in cartilage repair,” Arthritis research & therapy, vol. 16, no. 1, pp. 1-15, 2014.
[22] B. N. Brown and S. F. Badylak, “Extracellular matrix as an inductive scaffold for functional tissue reconstruction,” Translational Research, vol. 163, no. 4, pp. 268-285, 2014.
[23] S. L. Ding et al., “Microcarriers in application for cartilage tissue engineering: Recent progress and challenges,” Bioact Mater, vol. 17, pp. 81-108, November 2022, doi: 10.1016/j.bioactmat.2022.01.033.
[24] B. Li et al., “Past, present, and future of microcarrier-based tissue engineering,” J Orthop Translat, vol. 3, no. 2, pp. 51-57, April 2015, doi: 10.1016/j.jot.2015.02.003.
[25] D. Philp, S. S. Chen, W. Fitzgerald, J. Orenstein, L. Margolis, and H. K. Kleinman, “Complex extracellular matrices promote tissue-specific stem cell differentiation,” Stem cells, vol. 23, no. 2, pp. 288-296, 2005.
[26] A. J. Sutherland et al., “Decellularized cartilage may be a chondroinductive material for osteochondral tissue engineering,” PloS one, vol. 10, no. 5, p. e0121966, 2015.
[27] F. Albrecht, “Closure of joint cartilage defects using cartilage fragments and fibrin glue,” Fortschritte der Medizin, vol. 101, no. 37, pp. 1650-1652, 1983.
[28] J. Xue et al., “Repair of articular cartilage defects with acellular cartilage sheets in a swine model,” Biomedical Materials, vol. 13, no. 2, p. 025016, 2018.
[29] T. Gwisai, N. Mirkhani, M. G. Christiansen, T. T. Nguyen, V. Ling, and S. Schuerle, “Magnetic torque-driven living microrobots for increased tumor infiltration,” Science Robotics, vol. 7, no. 71, p. eabo0665, 2022.
[30] P. M. Price, W. E. Mahmoud, A. A. Al-Ghamdi, and L. M. Bronstein, “Magnetic drug delivery: where the field is going,” Frontiers in chemistry, vol. 6, p. 619, 2018.
[31] T. Vangijzegem, D. Stanicki, and S. Laurent, “Magnetic iron oxide nanoparticles for drug delivery: Applications and characteristics,” Expert opinion on drug delivery, vol. 16, no. 1, pp. 69-78, 2019.
[32] J. Li et al., “Development of a magnetic microrobot for carrying and delivering targeted cells,” Science Robotics, vol. 3, no. 19, p. eaat8829, 2018.
[33] T. Wei et al., “Development of Magnet-Driven and Image-Guided Degradable Microrobots for the Precise Delivery of Engineered Stem Cells for Cancer Therapy,” Small, vol. 16, no. 41, p. e1906908, October 2020, doi: 10.1002/smll.201906908.
[34] A.-G. Niculescu, C. Chircov, and A. M. Grumezescu, “Magnetite nanoparticles: Synthesis methods-A comparative review,” Methods, 2021.
[35] L. S. Ganapathe, M. A. Mohamed, R. Mohamad Yunus, and D. D. Berhanuddin, “Magnetite (Fe3O4) Nanoparticles in Biomedical Application: From Synthesis to Surface Functionalisation,” Magnetochemistry, vol. 6, no. 4, 2020, doi: 10.3390/magnetochemistry6040068.
[36] M. Unni, J. Zhang, T. J. George, M. S. Segal, Z. H. Fan, and C. Rinaldi, “Engineering magnetic nanoparticles and their integration with microfluidics for cell isolation,” Journal of colloid and interface science, vol. 564, pp. 204-215, 2020.
[37] A. Gholami, S. M. Mousavi, S. A. Hashemi, Y Ghasemi, W.-H. Chiang, and N. Parvin, “Current trends in chemical modifications of magnetic nanoparticles for targeted drug delivery in cancer chemotherapy,” Drug metabolism reviews, vol. 52, no. 1, pp. 205-224, 2020.
[38] X. Chen et al., “Electrospinning multilayered scaffolds loaded with melatonin and Fe₃O₄ magnetic nanoparticles for peripheral nerve regeneration,” Advanced Functional Materials, vol. 30, no. 38, p. 2004537, 2020.
[39] L. Xing, P. Liao, H. Mo, D. Li, and D. Sun, “Preformation Characterization of a Torque-Driven Magnetic Microswimmer With Multi-Segment Structure,” IEEE Access, vol. 9, pp. 29279-29292, 2021, doi: 10.1109/access.2021.3058527.
[40] F. Fan et al., “Rotating magnetic field-controlled fabrication of magnetic hydrogel with spatially disk-like microstructures,” Science China Materials, vol. 61, no. 8, pp. 1112-1122, 2018.
[41] D. Ayyildiz-Tamis, K. Avci, and S. I. Deliloglu-Gurhan, “Comparative investigation of the use of various commercial microcarriers as a substrate for culturing mammalian cells,” In Vitro Cell Dev Biol Anim, vol. 50, no. 3, pp. 221-31, March 2014, doi: 10.1007/s11626-013-9717-y.
[42] H. Tavassoli, S. N. Alhosseini, A. Tay, P. P. Y Chan, S. K. Weng Oh, and M. E. Warkiani, “Large-scale production of stem cells utilizing microcarriers: A biomaterials engineering perspective from academic research to commercialized products,” Biomaterials, vol. 181, pp. 333-346, October 2018, doi: 10.1016/j.biomaterials.2018.07.016.
[43] Y Liu, Q. Lian, J. He, J. Zhao, Z. Jin, and D. Li, “Study on the microstructure of human articular cartilage/bone interface,” Journal of Bionic Engineering, vol. 8, no. 3, pp. 251-262, 2011.
[44] H. Yin et al., “Functional tissue-engineered microtissue derived from cartilage extracellular matrix for articular cartilage regeneration,” Acta biomaterialia, vol. 77, pp. 127-141, 2018.
[45] A. Emami, T. Talaei-Khozani, Z. Vojdani, and N. Zarei Fard, “Comparative assessment of the efficiency of various decellularization agents for bone tissue engineering,” J Biomed Mater Res B Appl Biomater; vol. 109, no. 1, pp. 19-32, January 2021, doi: 10.1002/jbm.b.34677.
[46] H. Hartanto, M. Wu, M. L. Lam, and T.-H. Chen, “Microfluidic immunoassay for detection of serological antibodies: A potential tool for rapid evaluation of immunity against SARS-CoV-2,” Biomicrofluidics, vol. 14, no. 6, 2020.
[47] M. Wu, G. Wang, L. T. Chu, H. Huang, and T.-H. Chen, “Cascade-amplified microfluidic particle accumulation enabling quantification of lead ions through visual inspection,” Sensors and Actuators B: Chemical, vol. 324, p. 128727, 2020.
[48] X. N. Pham, T. P. Nguyen, T. N. Pham, T. T. N. Tran, and T. V. T. Tran, “Synthesis and characterization of chitosan-coated magnetite nanoparticles and their application in curcumin drug delivery,” Advances in Natural Sciences: Nanoscience and Nanotechnology, vol. 7, no. 4, p. 045010, 2016.
[49] M. G. Haugh and S. C. Heilshorn, “Integrating concepts of material mechanics, ligand chemistry, dimensionality and degradation to control differentiation of mesenchymal stem cells,” Current Opinion in Solid State and Materials Science, vol. 20, no. 4, pp. 171-179, 2016.
[50] J. E. Frith et al., “Mechanically-sensitive miRNAs bias human mesenchymal stem cell fate via mTOR signalling,” Nature communications, vol. 9, no. 1, p. 257, 2018.
[51] C. Chen et al., “Compression regulates gene expression of chondrocytes through HDAC4 nuclear relocation via PP2A-dependent HDAC4 dephosphorylation,” Biochimica et Biophysica Acta (BBA)-Molecular Cell Research, vol. 1863, no. 7, pp. 1633-1642, 2016.
[52] M. Kääb, I. Ap Gwynn, and H. Notzli, “Collagen fibre arrangement in the tibial plateau articular cartilage of man and other mammalian species,” The Journal of Anatomy, vol. 193, no. 1, pp. 23-34, 1998.
[53] C. R. Chu, M. Szczodry, and S. Bruno, “Animal models for cartilage regeneration and repair,” Tissue Engineering Part B: Reviews, vol. 16, no. 1, pp. 105-115, 2010.
[54] X. Nie, Y J. Chuah, W. Zhu, P. He, Y Peck, and D.-A. Wang, “Decellularized tissue engineered hyaline cartilage graft for articular cartilage repair,” Biomaterials, vol. 235, p. 119821, 2020.
[55] Z. Zhou, W. Wu, J. Fang, and J. Yin, “Polymer-based porous microcarriers as cell delivery systems for applications in bone and cartilage tissue engineering,” International Materials Reviews, vol. 66, no. 2, pp. 77-113, 2021.
[56] S. V. Madihally and H. W. Matthew, “Porous chitosan scaffolds for tissue engineering,” Biomaterials, vol. 20, no. 12, pp. 1133-1142, 1999.
[57] K. A. Athanasiou, M. Rosenwasser, J. Buckwalter, T. Malinin, and V. C. Mow, “Interspecies comparisons of in situ intrinsic mechanical properties of distal femoral cartilage,” Journal of orthopaedic research, vol. 9, no. 3, pp. 330-340, 1991.
[58] J. Malda and C. G. Frondoza, “Microcarriers in the engineering of cartilage and bone,” Trends in biotechnology, vol. 24, no. 7, pp. 299-304, 2006.
[59] P. Szpak, “Fish bone chemistry and ultrastructure: implications for taphonomy and stable isotope analysis,” Journal of Archaeological Science, vol. 38, no. 12, pp. 3358-3372, 2011.
[60] J. Brinckmann, H. Notbohm, and P. K. Müller, Collagen: primer in structure, processing and assembly. Springer Science & Business Media, 2005.
[61] L. J. Deleu, M. A. Lambrecht, J. Van de Vondel, and J. A. Delcour, “The impact of alkaline conditions on storage proteins of cereals and pseudo-cereals,” Current Opinion in Food Science, vol. 25, pp. 98-103, 2019.
[62] D. Meng et al., “The effect of alkaline pretreatment on the biochemical characteristics and fibril-forming abilities of types I and II collagen extracted from bester sturgeon by-products,” International journal of biological macromolecules, vol. 131, pp. 572-580, 2019.
[63] D. Liu et al., “Effects of alkaline pretreatments and acid extraction conditions on the acid-soluble collagen from grass carp (Ctenopharyngodon idella) skin,” Food Chemistry, vol. 172, pp. 836-843, 2015.
[64] S. Hattori, E. Adachi, T. Ebihara, T. Shirai, I. Someki, and S. Irie, “Alkali-treated collagen retained the triple helical conformation and the ligand activity for the cell adhesion via α2β1 integrin,” The journal of biochemistry, vol. 125, no. 4, pp. 676-684, 1999.
[65] T. FUJII, “The effect of amines added to an alkali-pretreatment on the solubilisation of collagen and on the properties of gelatin,” 1969.
[66] J. RAUTERBERG and K. KÜHN, “The renaturation behaviour of modified collagen molecules,” 1968.
[67] S. Jambhulkar, W. Xu, D. Ravichandran, J. Prakash, A. N. Mada Kannan, and K. Song, “Scalable alignment and selective deposition of nanoparticles for multifunctional sensor applications,” Nano Letters, vol. 20, no. 5, pp. 3199-3206, 2020.
[68] M. J. Sawkins et al., “Hydrogels derived from demineralized and decellularized bone extracellular matrix,” Acta Biomater; vol. 9, no. 8, pp. 7865-73, August 2013, doi: 10.1016/j.actbio.2013.04.029.
[69] G. Chen and Y Lv, “Decellularized Bone Matrix Scaffold for Bone Regeneration,” Methods Mol Biol, vol. 1577, pp. 239-254, 2018, doi: 10.1007/7651_2017_50.
[70] T. E. Ichim, P. O'Heeron, and S. Kesari, “Fibroblasts as a practical alternative to mesenchymal stem cells,” Journal of translational medicine, vol. 16, no. 1, pp. 1-9, 2018.
[71] D. Li, F. Niu, J. Li, X. Li, and D. Sun, “Gradient-Enhanced Electromagnetic Actuation System With a New Core Shape Design for Microrobot Manipulation,” IEEE Transactions on Industrial Electronics, vol. 67, no. 6, pp. 4700-4710, 2020, doi: 10.1109/tie.2019.2928283.
[72] L. Xing et al., “A New Drive System for Microagent Control in Targeted Therapy Based on Rotating Gradient Magnetic Fields,” Advanced Intelligent Systems, 2022, doi: 10.1002/aisy.202100214.
[73] L. Yang, J. Yu, S. Yang, B. Wang, B. J. Nelson, and L. Zhang, “A Survey on Swarm Microrobotics,” IEEE Transactions on Robotics, vol. 38, no. 3, pp. 1531-1551, 2022, doi: 10.1109/tro.2021.3111788.
[74] J. Yu, L. Yang, and L. Zhang, “Pattern generation and motion control of a vortex-like paramagnetic nanoparticle swarm,” The International Journal of Robotics Research, vol. 37, no. 8, pp. 912-930, 2018, doi: 10.1177/0278364918784366.
[75] P. Wrede et al., “Real-time 3D optoacoustic tracking of cell-sized magnetic microrobots circulating in the mouse brain vasculature,” Science Advances, vol. 8, no. 19, p. eabm9132, 2022.
[76] W. Qiu et al., “A Solid-Liquid Composite Lubricating “Nano-Snowboard” for Long-Acting Treatment of Osteoarthritis,” Advanced Functional Materials, vol. 32, no. 46, p. 2208189, 2022.
[77] J. F. Abulhasan and M. J. Grey, “Anatomy and physiology of knee stability,” Journal of Functional Morphology and kinesiology, vol. 2, no. 4, p. 34, 2017.
[78] J. Ferreira-Gomes, S. Adaes, and J. M. Castro-Lopes, “Assessment of movement-evoked pain in osteoarthritis by the knee-bend and CatWalk tests: a clinically relevant study,” The Journal of Pain, vol. 9, no. 10, pp. 945-954, 2008.
[79] D. H. Vrinten and F. F. Hamers, “‘CatWalk’automated quantitative gait analysis as a novel method to assess mechanical allodynia in the rat; a comparison with von Frey testing,” Pain, vol. 102, no. 1-2, pp. 203-209, 2003.
[80] X. Yan et al., “Multifunctional biohybrid magnetite microrobots for imaging-guided therapy,” Science robotics, vol. 2, no. 12, p. eaaq1155, 2017.
[81] R. S. Hennessy et al., “Supercritical carbon dioxide-based sterilization of decellularized heart valves,” JACC Basic Transl Sci, vol. 2, no. 1, pp. 71-84, February 2017, doi: 10.1016/j.jacbts.2016.08.009.
[82] E. Rieder et al., “Decellularization protocols of porcine heart valves differ importantly in efficiency of cell removal and susceptibility of the matrix to recellularization with human vascular cells,” The Journal of thoracic and cardiovascular surgery, vol. 127, no. 2, pp. 399-405, 2004.
[83] V. Samouillan et al., “Thermal analysis characterization of aortic tissues for cardiac valve bioprostheses,” Journal of Biomedical Materials Research: An Official Journal of The Society for Biomaterials, The Japanese Society for Biomaterials, and The Australian Society for Biomaterials and the Korean Society for Biomaterials, vol. 46, no. 4, pp. 531-538, 1999.
[84] E. Bodnar, E. Olsen, R. Florio, and J. Dobrin, “Damage of porcine aortic valve tissue caused by the surfactant sodiumdodecylsulphate,” The Thoracic and cardiovascular surgeon, vol. 34, no. 02, pp. 82-85, 1986.
[85] T.-X. Fan, S.-K. Chow, and D. Zhang, “Biomorphic mineralization: From biology to materials,” Progress in Materials Science, vol. 54, no. 5, pp. 542-659, 2009, doi: 10.1016/j.pmatsci.2009.02.001.
[86] Y Eguchi, S. Ohtori, M. Sekino, and S. Ueno, “Effectiveness of magnetically aligned collagen for neural regeneration in vitro and in vivo,” Bioelectromagnetics, vol. 36, no. 3, pp. 233-43, April 2015, doi: 10.1002/bem.21896.
[87] A. Pistone et al., “Engineering of Chitosan-Hydroxyapatite-Magnetite Hierarchical Scaffolds for Guided Bone Growth,” Materials (Basel), vol. 12, no. 14, Jul. 20 2019, doi: 10.3390/ma12142321.
[88] E. L. Mayes, F. Vollrath, and S. Mann, “Fabrication of magnetic spider silk and other silk-fiber composites using inorganic nanoparticles,” Advanced Materials, vol. 10, no. 10, pp. 801-805, 1998.
[89] M. Banobre-Lopez et al., “Hyperthermia Induced in Magnetic Scaffolds for Bone Tissue Engineering,” IEEE Transactions on Magnetics, vol. 50, no. 11, pp. 1-7, 2014, doi: 10.1109/tmag.2014.2327245.
[90] M. Stiehler, C. Bunger, A. Baatrup, M. Lind, M. Kassem, and T. Mygind, “Effect of dynamic 3-D culture on proliferation, distribution, and osteogenic differentiation of human mesenchymal stem cells,” J Biomed Mater Res A, vol. 89, no. 1, pp. 96-107, April 2009, doi: 10.1002/jbm.a.31967.
[91] N. Diomidis, S. Mischler, N. S. More, M. Roy, and S. N. Paul, “Fretting-corrosion behavior of 3 titanium alloys in simulated synovial fluid,” Wear, vol. 271, no. 7-8, pp. 1093-1102, 2011, doi: 10.1016/j.wear.2011.05.010.
[92] D. Davies, “Paper 7: properties of synovial fluid,” in Proceedings of the Institution of Mechanical Engineers, Conference Proceedings, 1966, vol. 181, no. 10: SAGE Publications Sage UK: London, England, pp. 25-29.

What is claimed is:

1. A tissue-derived implant comprising: a plurality of decellularized extracellular cartilage particles, wherein each of the plurality of cartilage extracellular particles comprise one or more magnetic particles and mesenchymal stem cells (MSCs).

2. The tissue-derived implant of claim 1, wherein the plurality of decellularized extracellular cartilage particles have an average diameter of 100-400 μm.

3. The tissue-derived implant of claim 1, wherein the plurality of decellularized extracellular cartilage particles have an average diameter about 238 μm.

4. The tissue-derived implant of claim 1, wherein the plurality of decellularized extracellular cartilage particles are derived from human, mouse, rat, dog, cat, rabbit, horse, pig, or nonhuman primate cartilage.

5. The tissue-derived implant of claim 1, wherein the plurality of decellularized extracellular cartilage particles are prepared by contacting a plurality of extracellular cartilage particles with a solution comprising sodium dodecyl sulfate thereby forming the plurality of decellularized extracellular cartilage particles.

6. The tissue-derived implant of claim 1, wherein the one or more magnetic particles have an average size of 100-300 nm.

7. The tissue-derived implant of claim 1, wherein the one or more magnetic particles have an average size about 200 nm.

8. The tissue-derived implant of claim 1, wherein the one or more magnetic particles comprise Fe₂O₃, Fe₃O₄, FeNi, FePt, Fe, or CoNi alloy.

9. The tissue-derived implant of claim 1, wherein the one or more magnetic particles have an average coating concentration on the plurality of decellularized extracellular cartilage particles of 2-20% wt/wt based on the weight of the one or more magnetic particles and a wet weight of the decellularized extracellular cartilage particles.

10. The tissue-derived implant of claim 1, wherein the one or more magnetic particles have an average coating concentration on the plurality of decellularized extracellular cartilage particles of about 10% wt/wt based on the weight of the one or more magnetic particles and a wet weight of the decellularized extracellular cartilage particles.

11. The tissue-derived implant of claim 1, wherein the MSCs comprise bone marrow mesenchymal stem cells.

12. The tissue-derived implant of claim 1, wherein the MSCs are present on a surface of each of the plurality of decellularized extracellular cartilage particles at seeding density of 300-3,000 MSCs per mm² of the plurality of decellularized extracellular cartilage particles.

13. The tissue-derived implant of claim 1, wherein the plurality of decellularized extracellular cartilage particles have an average diameter of 150-350 μm; the one or more magnetic particles comprise Fe₃O₄ and have average diameter of 100-300 nm; and the MSCs comprise bone marrow mesenchymal stem cells.

14. The tissue-derived implant of claim 1, wherein the plurality of decellularized extracellular cartilage particles have an average diameter about 238 μm; the one or more magnetic particles comprise Fe₃O₄ and have average diameter of about 200 nm; and the MSCs comprise bone marrow mesenchymal stem cells.

15. The tissue-derived implant of claim 14, wherein the MSCs are present on a surface of each the plurality of decellularized extracellular cartilage particles at seeding density of 300-3,000 MSCs per mm² of the plurality of decellularized extracellular cartilage particles.

16. A method of treating a cartilage defect in a subject in need thereof, the method comprising: administering a therapeutically effective amount of the tissue-derived implant of claim 1 to the subject.

17. The method of claim 16 further comprising exposing the tissue-derived implant to a magnetic field thereby directing the tissue derived implant to the cartilage defect after administering the tissue-derived implant to the subject.

18. The method of claim 17, wherein the magnetic field is a rotating magnetic field.

19. The method of claim 16 further comprising imaging the tissue-derived implant using ultrasound-guided photoacoustic imagining.
